(19) United States
(12) Patent Application Publication
(10) Pub. No.: US 2007/0037797 A1
(43) Pub. Date: Feb. 15, 2007

(54) Method of reducing the risk of adverse cardiovascular (CV) events associated with the administration of pharmaceutical agents which favor CV events

(76) Inventor: Harold Richard Hellstrom, Fayetteville, NY (US)

Correspondence Address: BAKER & BOTTS L.L.P., 30 ROCKEFELLER PLAZA, 44TH FLOOR, NEW YORK, NY, 10112-4498, US

(21) Appl. No.: 11/489,996

(22) Filed: Jul. 20, 2006

Related U.S. Application Data
(60) Provisional application No. 60/708,728, filed on Aug. 15, 2005.
(60) Provisional application No. 60/735,277, filed on Nov. 11, 2005.
(60) Provisional application No. 60/782,594, filed on Mar. 14, 2006.
(60) Provisional application No. 60/801,790, filed on May 19, 2006.

(51) Int. Cl. A61K 31/549; A61K 31/4152; A61K 31/401; A61K 31/366; A61K 31/365; A61K 31/22

(52) U.S. Cl. 514/223.5; 514/423; 514/406; 514/471; 514/460; 514/548

(57) ABSTRACT

Methods and compositions for reducing the risks of adverse cardiovascular (CV) events associated with the administration of pharmaceutical agents which induce or increase the risk of one or more adverse CV events, particularly the non-steroidal anti-inflammatory drugs (NSAIDs) and especially the cyclooxygenase-2 (COX-2) inhibitors, are disclosed. The methods involve the administration of compositions comprising the adverse CV event-inducing agent and additional pharmaceutical agents for reducing the risk of adverse CV events. In specific embodiments, the agent is selected from the group consisting of hydroxymethylglutaryl-coenzyme A reductase inhibitors (statins), angiotensin converting enzyme (ACE) inhibitors and angiotensin II receptor blockers (ARBs).

### CROSS-REFERENCE TO RELATED APPLICATIONS

This application claims priority to U.S. Patent Application Nos. 60/708,728 filed Aug. 15, 2005, 60/735,277 filed Nov. 11, 2005, 60/782,594 filed Mar. 14, 2006, and 60/801,790 filed May 19, 2006, each of which is incorporated by reference herein in its entirety.

### FIELD OF THE INVENTION

The present invention involves methods for coadministering pharmaceutical agents that can reduce or prevent the risk of myocardial infarction and/or other adverse cardiovascular (CV) events, with other pharmaceutical agents which facilitate the development of adverse CV events. The present invention also provides a program for reducing the risk of adverse CV events that involve coadministering CV-favoring pharmaceuticals with other pharmaceutical agents that can reduce or prevent the risk of adverse CV events in combination with healthy living habits.

### BACKGROUND OF THE INVENTION

Many individuals are at an elevated risk of suffering serious to life-threatening CV events, such as myocardial infarction (heart attack), cardiac arrest, congestive heart failure, stroke, peripheral vascular disease and/or claudication. The risk factors are numerous and widespread throughout the world population. They include diabetes, hypercholesterolemia (high serum cholesterol), hypertension, angina, systemic lupus erythematosus, cigarette smoking, prior heart attacks or strokes, hemodialysis, hyperhomocysteine levels, obesity, sedentary lifestyle, and others. Recently, there have been a number of reports associating COX-2 inhibitors and other non-steroidal anti-inflammatory drugs (NSAIDs), and other agents such as sympathomimetic agents (e.g., anti-attention deficit hyperactivity disorder (ADHD) agents), muraglitazar and acetaminophen, to an elevated risk of serious adverse CV events. Thus, the administration of, for example, certain NSAIDs, and especially the COX-2 inhibitors, is considered an additional risk factor that can lead to serious and life threatening CV problems.

### Non-Selective NSAIDs and COX-2 Inhibitors

A pathological role for the prostaglandins has been implicated in a number of human disease states, including rheumatoid and osteoarthritis, pyrexia, asthma, bone resorption, nephrotoxicity, atherosclerosis, hypotension, shock, pain, cancer, and Alzheimer disease. NSAIDs are widely used for the treatment of pain, inflammation, and acute and chronic inflammatory disorders. These compounds inhibit the activity of the enzyme cyclooxygenase (COX), also known as prostaglandin G/H synthase, which is the enzyme that converts arachidonic acid into prostanoids. The NSAIDs also inhibit the production of other prostaglandins, especially prostaglandin G2, prostaglandin H₂ and prostaglandin E₂, thereby reducing the prostaglandin-induced pain and swelling associated with the inflammation process.

Two forms of cyclooxygenases are now known, a constitutive isoform (COX-1) and an inducible isoform (COX-2), of which expression is upregulated at sites of inflammation (Vane et. al., Proc. Natl. Acad. Sci. USA, 1994, 91, 2046). COX-1 appears to play a physiological role and to be responsible for gastrointestinal and renal protection. On the other hand, COX-2 appears to play a pathological role and is believed to be the predominant isoform present in inflammatory conditions. The therapeutic use of conventional COX inhibitors (non-selective NSAIDS) is limited due to drug-associated side effects, including life threatening gastric ulceration and hemorrhage and renal toxicity. As a result, inhibitors specific for COX-2 have been developed and some are now on the market. These drugs maintain the ability to alleviate pain with reduced adverse gastrointestinal effects (Griswold et al. Med. Res. Rev. 16(2): 181-206 (1996); Lane Rheumatol. 24 (Suppl 49):20-4 (1997); Lipsky et al. J. Rheumatol. 24(Suppl 49):9-14 (1997)). See, for example, U.S. Pat. Nos. 5,393,790; 5,409,944; 5,418,254; 5,420,343; 5,436,265; 5,474,995; 5,476,944; 5,486,534; 5,510,368; 5,521,213; 5,536,752; 5,547,975; 5,550,142; 5,552,422; 5,565,482; 5,576,339; 5,580,985; 5,585,504; 5,593,994 and 5,596,008, all of which are incorporated herein by reference in their entirety.

A COX-2 inhibitor selectively inhibits the COX-2 form of the enzyme more than the COX-1 form. To be classified as a selective COX-2 inhibitor, a compound should inhibit COX-2 at least five times more than COX-1, or should have at least a 5:1 ratio of COX-2 to COX-1 inhibitory activity. Preferably, a COX-2 inhibitor should have an even greater selectivity than 5:1 for inhibiting COX-2, or from 5:1 to 100:1. A selective COX-2 inhibitor would be capable of producing a concentration level in the blood that would reduce pain by 80 to 90% by inhibiting COX-2, with little or no effect on the COX-1 form of the enzyme. Numerous studies have shown that the relative incidence of GI side effects from NSAIDs can be correlated to the relative COX-2 specificity of these anti-inflammatory agents. The higher the specificity for COX-2 over COX-1, the lower the incidence of GI upsets.

However, selectivity for COX-2 over COX-1 has recently been found to cause unwanted complications. Certain side-effects may result from COX inhibitors that are extremely selective for COX-2. For example, in the Adenomatous Polyp Prevention on Vioxx (APPROVe) study, which was designed to test the efficacy of rofecoxib on the prevention of benign sporadic colonic adenomas, a significant increase (approximately two times) in the incidence of serious thrombotic adverse events, including myocardial infarction, unstable angina, ischemic stroke, and peripheral vascular events, was shown for patients receiving 25 mg/day of rofecoxib versus placebo. Additionally, in the Vioxx Gastrointestinal Outcomes Research (VIGOR) trial, a significant increase (approximately two times) in the incidence of myocardial infarction was observed for rofecoxib (VIOXX®) versus the non-selective NSAID naproxen.

Further evidence of the increased incidence of adverse CV events upon administration of COX-2 inhibitors appeared in the National Cancer Institute's Adenoma Prevention with Celecoxib (APC) trial. In the APC trial, a 2-3 fold increase in adverse CV events was seen for celecoxib versus placebo after a mean duration of 33 months. The study also revealed a dose response relationship, with a hazard of 2.5 for celecoxib 200 mg twice daily and 3.4 for celecoxib 400 mg twice daily compared to placebo for the composite endpoint of death from CV causes.

More recent experimental evidence supports the findings of the COX-2 inhibitor trials with a probable mechanism of action causing adverse CV events (Mukherjee et al. JAMA 286:954-959 (2001); Mukherjee et al. Science 296:539-541 (2002)). An understanding of the physiologic features of COX isoenzymes has led to the appreciation that drugs that preferentially inhibit COX-2 may lead theoretically to problems in thrombosis, salt and water balance, and healing. The COX-1 enzyme is involved in the synthesis of thromboxane A₂, a compound responsible for vasoconstriction and platelet aggregation. In contrast, COX-2 promotes the production of prostacyclin, which leads to vasodilation and acts as a moderator of platelet aggregation. Elevated levels of thromboxane A₂ (relative to prostacyclin) may therefore contribute to a variety of CV events, including myocardial infarction and stroke. In a normal subject, the two enzymes are in a homeostatic balance; however, COX-2 selective inhibitors perturb this balance by only blocking the production of prostacyclin, while allowing thromboxane A₂ production to remain unchecked (Fitzgerald, Am. J. Cardiol. 89:26D-32D (2002). As a result, the COX-2 inhibitors increase vasoconstrictive events and platelet aggregation and, therefore, elevate the risk of adverse CV events. Furthermore, the higher a patient's intrinsic risk of CV disease, the more likely the hazards associated with the administration of a COX-2 inhibitor would manifest itself rapidly in the form of a clinical event (Fitzgerald, New Engl J. Med. 351:1709 (2004)).

Current pharmaceutical COX-2 inhibitors, such as valdecoxib, celecoxib and rofecoxib, are highly specific and would be expected to have very little, if any, COX-1 inhibitory activity at the doses used to reduce pain and inhibit COX-2 activity. Thus, the cardiac-related side effects that have been noted with the use of some COX-2 selective inhibitors may be related to the lack of any COX-1 inhibition while significantly inhibiting COX-2. According to 80% inhibitory concentration (IC₈₀) ratios of COX-2 relative to COX-1 in human whole blood assays, rofecoxib showed a selectivity ratio of 80:1 and celecoxib a selectivity ratio of 9:1 (Vane et al. Proc. Natl. Acad. Sci. 1999; 96:7563-8).

COX-2 inhibitors have been marketed since 1999 as safer alternatives to nonsteroidal anti-inflammatory drugs (NSAIDs). However, debate about their cardiac safety has culminated in the recent withdrawal of rofecoxib (VIOXX®) and valdecoxib (BEXTRA®) from the U.S. pharmaceutical market. On the other hand, celecoxib (CELEBREX®) has been allowed to remain available with a prescription because it has shown a lower incidence of adverse CV events in clinical trials. Several researchers have postulated that the degree of risk of adverse CV events associated with the COX-2 inhibitors can be correlated to the degree of COX-2 inhibitory activity of a pharmaceutical agent relative to its COX-1 inhibitory activity. Accordingly, the fact that rofecoxib is 10 times more selective for COX-2 than celecoxib may explain the observed difference in risk between the two inhibitors because the unopposed prothrombotic activity of thromboxane A₂ associated with rofecoxib is likely to be considerably greater than that of celecoxib (Warner et al. Proc. Natl. Acad. Sci. USA 1999; 96:7563-8; Wright, CMAJ2002; 167:1131-7).

According to a Decision Memo for the Recommendations of Agency Action for COX-2 Selective and Non-Selective NSAIDs (Issued Apr. 6, 2005), the FDA has determined that three COX-2 inhibitors (i.e., rofecoxib, valdecoxib, and celecoxib) are all associated with an increased risk of adverse CV events compared to placebo.

According to the FDA, the available data representing the association COX-2 inhibitors with adverse CV events are best interpreted as a class effect for COX-2 selective and non-selective NSAIDs. Thus, all NSAIDs are currently thought to pose risk for adverse CV events.

There was no immediate appreciation that NSAIDs, particularly COX-2 inhibitors, would cause adverse CV events. Initially, because of their anti-inflammatory properties, these drugs were regarded useful in the treatment of CV disease.

For example, WO 98/47509 discloses the use of COX-2 inhibitors in the prevention of various CV disorders, including myocardial infarction (MI). The publication additionally discloses that the COX-2 inhibitors can be used alone or in combination with statins, ACE inhibitors, and aspirin, and that prevention of CV disorders with a COX-2 inhibitor is based on the fact that COX-2 inhibitors have anti-inflammatory properties. Because inflammation has been identified as a risk factor for CV disease and adverse CV events such as MI, Roniker et al. infer that COX-2 inhibitors have a beneficial effect in the prevention of CV diseases. There is no mention within the document of the risks of seriously adverse CV events associated with the COX-2 inhibitors or other non-selective NSAIDs. This method differs from that of the present invention because the combination of a COX-2 inhibitor with statins, ACE inhibitors, and/or aspirin in WO 98/47509 is considered to have a synergistic effect on the prevention of adverse CV events.

U.S. Pat. No. 6,323,226 is directed to the use of COX-2 inhibitors alone or in combination with other preventative drugs in the treatment or prevention of heart disease. Specifically, the patent discloses the administration of a COX-2 inhibitor in conjunction with ACE inhibitors and/or ARBs. The manuscript additionally discloses that treatment of heart disease with a COX-2 inhibitor is based on the fact that CHF occurs though mechanisms associated with, inter alia, inflammatory responses involving COX-2 activity. Thus, the patent teaches COX-2 inhibitors to be preventative with regard to the treatment of CHF. There is no mention within the document of the risks of serious adverse CV events associated with the COX-2 inhibitors or other non-selective NSAIDs. This method differs from that of the present invention because the combination of a COX-2 inhibitor with ACE inhibitors and/or ARBs in U.S. Pat. No. 6,323,226 is considered to have a synergistic effect on the prevention of adverse CV events.

US Published Application 2005/0020657 is directed to pharmaceutical compositions comprising a COX-2 inhibitor and a thromboxane A2 receptor antagonist and provides a method for avoiding the CV risks associated with COX-2 inhibitors by inhibiting the action of thromboxane A2. The Brunner publication discloses specifically blocking expression of thromboxane A2 by a thromboxane A2 receptor antagonist. Because COX-2 inhibitors preferentially block prostacyclin, leaving thromboxane A2 relatively unopposed, thromboxane A2 becomes relatively dominant with COX-2 inhibitor therapy. By blocking thromboxane with a thromboxane A2 receptor antagonist, Brunner asserts that the compositions described therein lessen the risk of adverse CV events. This invention follows the standard model for risk prevention as it targets the specific mechanism that is associated with the CV risks associated with the COX-2 inhibitors (i.e., elevated levels of thromboxane A2). This method differs from the present invention as it does not address an overall risk/prevention balance wherein the risks associated with the administration of COX-2 inhibitors can be reduced by factors that do not directly impact the expression of thromboxane A2.

It is an object of the present invention to reduce the risk of adverse CV events associated with the administration of NSAIDS, especially the COX-2 inhibitors, and other pharmaceuticals such as sympathomimetic agents (e.g., ADHD medications), muraglitazar and acetaminophen, with the co-administration of one or more pharmaceutical agents for reducing the risk of adverse CV events.

### SUMMARY OF THE INVENTION

In accordance with the present invention the CV risks associated with the administration of one or more first pharmaceutical agents, can be reduced or prevented by co-administering said first agent or agents with one or more preventative pharmaceutical agents for reducing the risk of adverse CV events. In some embodiments, the first pharmaceutical agent is selected from NSAIDs, COX-2 inhibitors, sympathomimetic agents, and acetaminophen, and the preventative pharmaceutical agent can be selected from the group consisting of hydroxymethylglutaryl-coenzyme A reductase inhibitors (“statins”), angiotensin converting enzyme (ACE) inhibitors and angiotensin II receptor blockers (ARBs).

The present invention also provides a program for reducing the risk of adverse CV events associated with the administration one or more first pharmaceutical agents, that involves (1) coadministering said first pharmaceutical agent or agents with one or more preventative pharmaceutical agents that can reduce or prevent the risk of adverse CV events; and/or (2) recommending that a patient in need of therapy with the first pharmaceutical agent or agents practice healthy living habits.

The present invention is directed to methods for reducing the risk of an adverse CV event in a patient to be treated with one or more first pharmaceutical agents which induce or increase the risk of an adverse CV event, the method comprising co-administering pharmaceutical combination comprising:
(a) a therapeutically effective amount of one or more first pharmaceutical agents; and
(b) one or more preventative pharmaceutical agents for reducing the risk of CV events;
wherein the method results in a lower overall risk for an adverse CV event compared to administration of the one or more first pharmaceutical agents without the one or more preventative pharmaceutical agents;
with the proviso that the preventative pharmaceutical agent is not a thromboxane A2 antagonist. In other embodiments, repeated co-administration is carried out for at least ten days, two weeks, ten weeks, three months, ten months, or two years. The repeated co-administration can be daily co-administration, every other day, or weekly co-administration, for example.

The present invention also provides a method for treating a chronic disorder with one of more first pharmaceutical agents that induce or increase the risk of an adverse CV event, while reducing the patient's risk of an adverse CV event, the method comprising repeatedly co-administering:
(a) a therapeutically effective amount of one or more first pharmaceutical agents; and
(b) an adverse CV event risk reducing amount of one or more preventative pharmaceutical agents; for at least two days.

The present invention also provides a method for designing a pharmaceutical combination comprising one or more first pharmaceutical agents and one or more preventative pharmaceutical agents, wherein the administration of the pharmaceutical combination to a patient in need of the one or more first pharmaceutical agents results in a lower overall risk for an adverse CV event as compared to the administration of the one or more first pharmaceutical agents alone, the method comprising
(1) determining that the one or more first pharmaceutical agents increase the risk of an adverse CV event; and
(2) combining a therapeutically effective amount of the one or more first pharmaceutical agents with an adverse CV event risk reducing amount of one or more preventative pharmaceutical agents.

The present invention thus addresses the need to, inter alia, provide the benefits of a first pharmaceutical agent or agents, such as pain relief, to a patient in need thereof with reduced risks of adverse CV events that are associated with the first pharmaceutical agent or agents.

### DETAILED DESCRIPTION

The present invention advantageously provides for reducing the risk of adverse CV events with the administration of a first pharmaceutical agent, for any purpose, e.g., analgesia, anti-inflammatory effect, antipyretic effect, treatment of attention deficit hyperactivity disorder (ADHD), and the like. Risk factor reduction, that is, eliminating risk factors of adverse CV events is the standard of medicine at the present time. However, in the case of, for example, NSAIDs and particularly COX-2 inhibitors, risk factor reduction requires elimination of these valuable drugs, so risk factor reduction is an undesirable way to reduce the incidence of adverse CV events. Based, in part, on analysis of data from hundreds if not thousands of research papers, it has now been discovered that risk factor reduction is not the only way to reduce the risk of adverse CV events

For example, the insights gained from studying years of literature reports have resulted in the discovery that combining administration of one or more first pharmaceutical agents, including NSAIDS and sympathomimetic agents, with one or more pharmaceutical agents for reducing the risk of adverse CV events surprisingly will reduce the risk of adverse CV events from the first agent. In a particular embodiment, the invention is based on the discovery that administering a COX-2 inhibitor, such as celecoxib (CELEBREX®), with a statin, such as LIPITOR®, permits treatment of pain and inflammation with reduced risk of developing an adverse CV event. The addition of one or more preventative agents will further reduce the risk of CV events.

The risk of adverse CV events associated with the administration of one or more first pharmaceutical agents, including NSAIDS and sympathomimetic agents, and especially a COX-2 inhibitor, will be further reduced by recommending that a patient in need of therapy associated with the first pharmaceutical agent undertake a total program comprising (1) the co-administration of the first agent or agents with one or more pharmaceutical agents for reducing the risk of adverse CV events, and (2) following a recommendation to begin healthier living habits including, but not limited to, maintaining proper weight, exercise, stress reduction, and healthy dietary habits.

### Definitions

The term “adverse cardiovascular (CV) event,” or simply “cardiovascular (CV) event,” as used herein refers, generally, to a disorder or disease of the cardiovascular system resulting from progressive vascular damage. Although the event may have a rather sudden onset, it can also refer to a progressive worsening of such a disorder or disease. Examples of adverse CV events include, without limitation: claudication, hypertension, cardiac arrest, myocardial infarction, ischemia, stroke, transient ischemic attacks, worsening of angina, congestive heart failure, left ventricular hypertrophy, sudden cardiac death, arrhythmias, thromboembolism and arterial and venous thromboses. Examples of progressive vascular diseases are those that affect the cerebral, coronary, renal, or peripheral circulations.

The terms “first pharmaceutical agent,” “pharmaceutical agent which induces or increases the risk of an adverse CV event,” and “CV-favoring agent” are synonymous and refer to any pharmaceutical agent that induces or increases the risk of inducing an adverse CV event. As non-steroidal anti-inflammatory drugs (NSAIDs) are the most common cause of pharmaceutical-induced CV events, this present invention will use NSAIDs, especially COX-2 inhibitors, as the prototype first pharmaceutical agents. Discussions of the methods, compositions and programs of the present invention using NSAIDs and COX-2 inhibitors, however, can be applied to all other first pharmaceutical agents, such as selective estrogen receptor modulators (SERMs), such as raloxifene, sympathomimetic agents, for example anti-ADHD pharmaceutical agents, acetaminophen and muraglitazar, which induce or increase the risk of inducing adverse CV events.

The terms “preventative factors” and “preventative measures,” as used herein, are synonymous and refer to any preventative measure that may be taken to reduce the risk of an adverse CV event. Preventative measures of the present invention include preventative measures for reducing the risk of adverse CV events associated with a first pharmaceutical agent, particularly an NSAID and especially a COX-2 inhibitor, such as pharmaceutical agents for reducing the risk of adverse CV events and/or healthy living habits.

The term “unit dose” or “unit dose form” refers to a single drug administration entity. By way of example, a single tablet, capsule, dragee, vial for injection or syringe combining both an NSAID, especially a COX-2 inhibitor, and at least one of a pharmaceutical agent for reducing the risk of CV events would constitute a unit dose form.

By “pharmaceutically acceptable,” such as in the recitation of a “pharmaceutically acceptable carrier,” or a “pharmaceutically acceptable acid addition salt,” is meant herein a material that is not biologically or otherwise undesirable, i.e., the material may be incorporated in a pharmaceutical composition administered to a patient without causing undesirable biological effects or interacting in a deleterious manner with any of the other components of the composition in which it is contained. “Pharmacologically active” (or simply “active”), as in a “pharmacologically active” derivative or metabolite, refers to a derivative or metabolite having the same type of pharmacological activity as the parent compound and approximately equivalent in degree. When the term “pharmaceutically acceptable” is used to refer to a derivative (e.g., a salt) of an active agent, it is to be understood that the compound is pharmacologically active as well, i.e., therapeutically effective to reduce elevated CV risk.

“Carriers” or “vehicles” as used herein refer to conventional pharmaceutically acceptable carrier materials suitable for drug administration, and include any such materials known in the art that are nontoxic and do not interact with other components of a pharmaceutical composition or drug delivery system in a deleterious manner.

The terms “effective amount” and “therapeutically effective amount” of a drug or pharmacologically active agent are synonymous and refer to a nontoxic but sufficient amount of the drug or agent to provide the desired effect. In the combination therapy of the present invention, an “effective amount” of one component of the combination is the amount of that component that is effective to provide the desired effect when used in combination with the other components of the combination. The amount that is “effective” will vary from subject to subject, depending on the age and general condition of the individual, the particular active agent or agents, and the like. Thus, it is not always possible to specify an exact “effective amount.” However, an appropriate “effective” amount in any individual case may be determined by one of ordinary skill in the art using routine experimentation.

The terms “treating” and “treatment” as used herein refer to reduction in severity and/or frequency of symptoms, elimination of symptoms and/or underlying cause, prevention of the occurrence of symptoms and/or their underlying cause, and improvement or remediation of damage. Thus, for example, “treating” a patient involves prevention of a particular disorder or adverse physiological event in a susceptible individual as well as treatment of a clinically symptomatic individual. In the context of the present invention, treatment refers particularly to the reduction of the risk of an adverse CV event(s) associated with the administration of one or more first pharmaceutical agents, for example, an NSAID, especially a COX-2 inhibitor.

The phrase “combination therapy,” in defining use of a first pharmaceutical agent, for example, an NSAID and especially a COX-2 inhibitor, with one or more preventative pharmaceutical agents for reducing the risk of CV events associated with the first agents is intended to embrace administration of each agent in a sequential manner in a regimen that will provide beneficial effects of the drug combination, and is also intended to embrace co-administration of the pharmaceutical agents in a substantially simultaneous manner, such as in a single capsule having a fixed ratio of active ingredients (i.e., a unit dose) or in multiple, separate capsules for each antagonist agent.

The terms “cardiovascular (CV) risk,” “elevated cardiovascular (CV) risk” and “cardiovascular (CV) risk factor” as used herein refer to an increased risk of incurring an adverse CV event, peripheral vascular disease, coronary heart disease, restenosis, or atherosclerosis in an individual, such risk being due to the administration of a first pharmaceutical agent, for example, an NSAID and especially a COX-2 inhibitor; and standard risk factors such as disorders, diseases, genetic factors, behaviors, diets, or other conditions or factors. The conditions or factors that lead to elevated CV risk include, without limitation: administration of a first pharmaceutical agent, for example, an NSAID and especially a COX-2 inhibitor; and standard risk factors such as systemic lupus erythematosus, current or prior cigarette smoking, diabetes, hypertension, stroke, angina, hemodialysis, receiving an organ transplant, manifest coronary artery disease, history of myocardial infarction, history of transient ischemic attacks or stroke, history of peripheral vascular disease, angina, hypertension, hypercholesterolemia, obesity, atherosclerosis, kidney disease, Chlamydia infection, Bartonella infection, and obstructive pulmonary disease.

The term “overall risk” for an adverse CV event, as used herein, refers to the total risk of an adverse CV event in a patient, said total risk being associated with administration of one or more of a first pharmaceutical agent, and/or one or more standard risk factors as set forth herein. Thus the overall risk is a risk due to the combination of the risk associated with the first pharmaceutical agent and the risks associated with standard risk factors. The overall risk for an adverse CV event can be lowered through the practice of the methods of the present invention through administration of a pharmaceutical agent for lowering the risk of an adverse CV event and/or the practice of healthy living habits.

The present invention is particularly pertinent to, for example, (1) the reduction of CV risks associated with the administration of a first pharmaceutical agent, particularly an NSAID, when the patient in need thereof is otherwise healthy and (2) the reduction of CV risks associated with the administration of a first pharmaceutical agent, particularly an NSAID, when the patient in need thereof possesses additional conditions or factors that lead to increased CV risk.

The terms “otherwise healthy,” “healthy patient,” and “healthy individual,” as used herein, refer to a patient who has no complication other than that which requires therapy with a first pharmaceutical agent as defined herein, for example an NSAID and especially a COX-2 inhibitor. Thus a patient who is otherwise healthy is only at an elevated risk for an adverse CV event due to the administration of the first pharmaceutical agent or agents.

“Reducing the risk” or “reduction of risk” of occurrence of adverse CV events refers to lowering the overall risk of occurrence of any of the conditions or factors set forth herein, in a patient at risk to developing the conditions such as, for example, a patient undergoing COX-2 inhibitor therapy.

The term “sympathomimetic agent” refers to class of pharmaceutical agents whose properties mimic those of a stimulated sympathetic nervous system. As such they increase cardiac output, dilate bronchioles, and usually produce constriction of blood vessels. Sympathomimetic agents elicit physiological responses similar to those produced during adrenergic nerve activity and, thus, are a are also known as adrenergic agents, adrenomimetic agents, and sympathetic agents. Within the class of sympathomimetic agents are the sympathomimetic amines including, but not limited to, amphetamine, dextroamphetamine, methamphetamine, benzphetamine, phentermine, chlorphentermine, fenfluramine, dextrofenfluramine, clortermine, mephentermine, phenmetrazine, phendimetrazine, diethylpropion, mazindol, phenylpropanolamine, ephedrine, pseudoephedrine and methylphenidate.

The term “therapeutically effective” means that sufficient drug is present to generate the therapeutic action for which the drug is given. For example, if a patient is being treated for pain then a “therapeutically effective” amount of a COX-2 inhibitor would be a dosage sufficient to reduce the severity or duration of the pain. If the patient is being treated for inflammation, then enough drug would need to be present to reduce the associated pain or swelling.

The terms “pharmaceutical agent for reducing the risk of cardiovascular (CV) event(s)” and “preventative pharmaceutical agent” as employed herein, are synonymous, and refer to any pharmaceutical agent that is known in the art to reduce the risk of the CV events set forth herein. Pharmaceutical agents for reducing the risk of CV event(s) include the HMG-CoA reductase inhibitors (statins), the angiotensin converting enzyme (ACE) inhibitors, and the angiotensin II receptor blockers (ARBs), particularly the HMG-CoA reductase inhibitors (statins). Additional pharmaceutical agents for reducing the risk of adverse CV events include agents that improve dyslipidemia as reported in the National Cholesterol Education Program (NCEP), Adult Treatment Panel (ATP) III (NIH Publication No. 02-5215, September 2002; Circulation 2002; 106; 3143-3421). The NCEP ATP III lists a number of agents for treating dyslipidemia including, bile acid sequestrants, for example, cholestyramine, colestipol, and colesevelam; nicotinic acid; fibric acid derivatives, for example, gemfibrozil, fenofibrate, and clofibrate; n-3 fatty acids; and hormone replacement therapy (HRT), for example, estrogen and progesterone. Other pharmaceutical agents for reducing the risk of CV events include inhibitors of the renin-angiotensin system other than ACEs and ARBs, peroxisome proliferator-activated receptor antagonists, and selective inhibitors of intestinal cholesterol, for example, ezetimibe. Additional preventative pharmaceutical agents include vasodilators (especially calcium channel blockers), PPAR agents, and thiazides. Pharmaceutical agents for reducing the risk of adverse CV events include combination therapies, for example a combination of a selective inhibitor of intestinal cholesterol (e.g., ezetimibe) and a statin (e.g., simvastatin) (VYTORIN®), and a combination of an inhibitor of cholesteryl ester transferase (e.g., torcetrapid) and a statin (e.g., LIPITOR®).

The term “determining that the one or more first pharmaceutical agents increase the risk of an adverse CV event,” as used herein, refers to any method that may be used to indicate an increased risk of an adverse CV events. These methods include, but are not limited to, analysis of the data provided in clinical trails and/or animal studies, analysis of the FDA's Adverse Event Reporting System (AERS), analysis of authoritative articles in the medical literature, a determination that the administration of a pharmaceutical agent of combination of agents leads to an increase in systolic blood pressure, and correlating pharmaceutical agents known to increase the risk of an adverse CV event with other pharmaceuticals known to operate by the same, or similar, mode of action (i.e., recognition that a number of drugs in a pharmaceutical class (e.g., sympathomimetic agents) increase the risk of adverse CV events is taken as evidence that other drugs in that class would also reasonably increase the risk of adverse CV events). The level of increase in systolic blood pressure which constitutes an increased risk for an adverse CV event is related to the total risk burden. For example, it is known that a pharmaceutical agent that increases systolic pressure by 5 millimeters mercury (mm) or more, especially during long term therapy, leads to an increase of morbidity and mortality (Nissen, S. E. NEJM 2006; 354:1445). However, even a minimal systolic blood pressure rise (e.g., 2 mm systolic) would increase the level of adverse CV events in those with multiple risk factors, such as dyslipidemia, obesity, etc.

The term “adverse CV event risk reducing amount of one or more preventative pharmaceutical agents,” as used herein, refers to an amount of the preventative agent sufficient to counteract (i.e., significantly lower) the risks of an adverse CV event associated with the one or more first pharmaceutical agents as defined herein.

Pharmaceutical agents directly designed to emulate helpful effects of a parasympathetic homeostatic shift are also included in the invention. Some examples are pharmaceutical agents directly designed to increase levels of nitric oxide (NO). NO release drugs have been used in IHD for more than a century (Behrends Curr Med Chem 2003; 10:291-301), and a variety of pharmacological NO prodrugs have been developed (Liu et al. Toxicology 2005; 208:289-297). Examples of NO donors are NCX 4016 (Emanueli et al. Arterioscler. Thromb. Vasc. Biol. 2004; 24:2082-2087) and S-nitrosylated captopril (Jia et al. Br. J. Pharmacol. 2001; 134:1698-1704).

### The Risk/Prevention Balance and Altered Homeostatic Theory

The current and standard methods for preventing ischemic heart disease (IHD), hypertension, and other adverse CV events are based solely on removal or reduction of risk factors. As evidence, risk factors for IHD are separated into two groups, modifiable and unmodifiable. Modifiable risk factors are hypertension, smoking, diabetes, obesity, physical activity, and atherogenic diet. Non-modifiable risk factors are age, male sex, and family history of premature IHD (Expert Panel on Detection, Evaluation, and Treatment of High Blood Cholesterol in Adults. Detection, evaluation, and treatment of high blood cholesterol in adults (Adult Treatment Panel III): third report of the National Cholesterol Education Program (NCEP) Expert Panel. Circulation 2002; 106:3145-3421). As might be expected, discussion of risk factors in this report was limited to the modifiable group, and the apparent impossibility of modifying the unmodifiable risk factor of age no doubt has limited interest in preventing disease in older individuals.

The prevention of, for example, IHD solely by risk factor reduction (i.e., the standard method) is likely based on the premise that multiple risk factors for IHD operate by individual mechanisms. It might seem reasonable that diverse risk factors as dyslipidemia, emotional stress, cold exposure, and hypertension have individual mechanisms; if each risk factor has its own mechanism, the only way to reduce the risk of IHD is to remove individual risk factors.

In contrast to this apparent standard view for the prevention of CV events is the prevention of CV events based on the principle of favorably altering the total balance of risk and preventative factors—a risk/prevention balance. Without wishing to be bound by theory, the principle of improving the overall risk/prevention balance is based on the principle that risk factors operate by a single (if complex) mechanism and are opposed by a single (if complex) mechanism of preventative factors. Also, it is asserted that prevention of disease based on favorably altering the overall risk/prevention balance more accurately reflects basic mechanisms of, for example, IHD and hypertension; if so, prevention based on this approach should be more effective.

This hypothesis first asserted that IHD and hypertension have a common basic mechanism of altered homeostasis (See, Hellstrom H R. Med Hypotheses. 1999; 53:194-199 and Hellstrom H R. Med Hypotheses. 2003; 60:12-25) and now is also applied to diabetes. The hypothesis avers that risk factors shift homeostasis toward disease, and that preventative factors shift homeostasis toward health; more specifically, risk factors cause inappropriate acute and/or chronic activation of sympathetic defensive action (fight/flight), and preventative factors shift homeostasis beneficially toward parasympathetic rest (conservation/withdrawal). The autonomic nervous system involves basic functions including vascular, nervous system, and metabolic function, and shifts of homeostasis are considered to include major mechanisms of these systems (Lefkowitz R J, Hoffman B B, Taylor P. “Neurotransmission: the autonomic and somatic motor nervous systems.” In: Hardman J G, Limbird L E, Molinoff P B, Ruddon R W, Gilman A G, editors. The Pharmacological Basis of Therapeutics. New York: McGraw-Hill, 1996: 105-139). Thus, without wishing to be bound by theory, if risk factors operate by sympathetic activation and preventative factors by parasympathetic activation, then risk and preventative factors operate by a single (if complex) opposing mechanism.

The position that risk and preventative factors operate by a single (if complex) opposing mechanism carries with it a major implication. If risk factors operate by a single mechanism and are opposed by preventative factors operating through an opposing mechanism, any preventative factor can oppose any risk factor and favorably alter the risk/prevention balance; as any risk factor can be opposed, this position allows for attenuation of unmodifiable risk factors such as age. This position also allows for attenuation of risk factors associated with the administration of a first pharmaceutical agent or agents, such as use of NSAIDs, especially COX-2 inhibitors, for various therapeutic purposes without the need to discontinue therapy with the first pharmaceutical agent or agents (i.e., remove the risk factor).

The view that inappropriate homeostatic shifts causing disease involve sympathetic activation, and that beneficial shifts involve parasympathetic activation, can be used to support the idea of a risk/prevention balance. The autonomic nervous system is known to operate through balance of sympathetic and parasympathetic activity (Lefkowitz R J, et al. In: The Pharmacological Basis of Therapeutics. New York: McGraw-Hill, 1996: 105-139) and a like balance is proposed for risk and preventative factors, i.e., a risk/prevention balance.

The theory has major similarities to other proposals. Favorably, sympathetic activation has been implicated in IHD, hypertension, and diabetes, and common mechanisms for these disorders have been proposed. However, the hypothesis underlying the present invention appears unique in its position that risk and preventative factors operate in a dynamic balance though shifts of homeostasis.

The hypothesis can be regarded as a modification and expansion of previous proposals. Both Selye's stress syndrome (Selye H. The Stress of Life: Revised Edition. New York: McGraw-Hill Book Co, 1978 and McEwen B S. N Engl J. Med. 1998; 338:171-179) and the altered homeostatic theory give a basic role to sympathetic hyperactivity and have been applied to IHD, hypertension, and diabetes. Also, IHD (Remme W J. Eur Heart J. 1998; 19(Suppl F):F62-F71) and hypertension (Julius S. Clin Exp Hypertens. 1995; 17:375-386) have been attributed to inappropriate activation of the sympathetic nervous system and to activation of fight/flight, and diabetes has been attributed to sympathetic activation (M, Rumantir M, Wiesner G et al. Am J Hypertens. 2001; 14:304S-309S) and to stress induced disturbance of the hypothalmic-pituitary-adrenal axis—which includes sympathetic activation (Rosmond R. Med Sci Monit. 2003; 9:RA35-RA39).

The altered homeostatic theory also is similar to proposals of common pathogenesis involving IHD, hypertension, and/or diabetes. As examples, IHD and diabetes have been related to inflammation (Ridker P M. Circulation. 2002; 105:2-4 and Pickup J C. Diabetes Care. 2004; 27:813-823) hypertension and IHD have been ascribed to the defense reaction (Julius S. Clin Exp Hypertens. 1995; 17:375-386), and the metabolic syndrome with its clustering of IHD, hypertension, diabetes, dyslipidemia, a thrombotic tendency, and obesity has been attributed to sympathetic activation and insulin resistance (Reaven G M, Lithell H, Landsberg L. N Engl J. Med. 1996; 334:374-381).

Table 1, below, summarizes the associations of attributes of sympathetic fight/flight with risk factors for IHD, hypertension, and diabetes. The superscripts in the table reference the publications, listed at the end of the specification, in which the associations between sympathetic fight/flight and risk factors have been reported for each particular entry.

As evidenced by the data represented in Table 1, risk factors are considered to operate by a single mechanism involving sympathetic activation. Twelve separate and diverse risk factors for IHD, hypertension, and diabetes, most of which are major, express sympathetic activation, and this provides evidence that risk factors operate by the single mechanism which involves sympathetic activation.

There is further evidence that risk factors are involved with sympathetic activation. Namely, major attributes of sympathetic activation are equivalent to four major risk factors for IHD, hypertension, and diabetes. As indicated in Table 1, sympathetic fight/flight prompts endothelial dysfunction (a thrombotic and vasoconstrictive tendency See, Verma S, Anderson T J. Circulation. 2002; 105:546-549), and increases levels of lipids, inflammation, and glucose. These four attributes of sympathetic fight/flight are equivalent to the risk factors for IHD, hypertension, and diabetes of endothelial dysfunction, dyslipidemia, inflammation, and insulin resistance (Table 1).

Without wishing to be bound by any particular theory, it is assumed that defensive sympathetic activation, when inappropriate and prolonged, can cause disease. In an acute fight/flight situation, activating the sympathetic nervous system and its associated components permits a more effective defense and can be lifesaving. Raising levels of lipids and glucose fuels the defense; activating a tendency toward thrombosis and vasoconstriction protects against exsanguination after injury (i.e., the hemostatic response), raising blood pressure (i.e., vasoconstriction) and heart rate enhances response capabilities, and activating inflammation protects against injury-induced infections. However, inappropriate and prolonged sympathetic activation is considered to change these helpful components of sympathetic activation to sympathetic-related risk factors of dyslipidemia, insulin resistance, endothelial dysfunction, and inflammation.

The sympathetic nervous system is highly interrelated, and reducing one of these risk factors/attributes of sympathetic activation, for example, reducing cholesterol values, would result in reduction of other risk factors and foster a shift from sympathetic activation to beneficial parasympathetic activation. Also, through parasympathetic activation, improving dyslipidemia, for example, will also have the helpful action of improving other sympathetic risk factors as inflammation, endothelial dysfunction, and glucose intolerance.

TABLE 1
Associations of Risk Factors with IHD, Hypertension, and Diabetes, and with Sympathetic Activation

| | | | | Sympathetic | | Attributes of Sympathetic Activation | | | | |
|---|---|---|---|---|---|---|---|---|---|---|
| | | | | Activation | | (Which also are Major Risk Factors | | | | |
| | Disorders | | | ↑ Sym- | ↓ Para- | | | | | ↑ Glucose/ |
| | | Hyper- | | pathetic | sympathetic | Endothelial | Thrombotic | ↑ Lipids/ | Inflam- | Insulin |
| | IHD | tension | Diabetes | Activity | activity | dysfunction* | tendency | Dyslipidemia | mation | resistance |
| Sympathetic Fight/ | | | | +¹ | +¹ | +² | +³ | +⁴ | +⁴ | +†³ |
| Flight and its | | | | | | | | | | |
| Attributes | | | | | | | | | | |
| Sympathetic | +⁵ | +⁶ | +⁷ | | | | | | | |
| Hyperactivity | | | | | | | | | | |
| “Sympathetic” | | | | | | | | | | |
| Risk Factors | | | | | | | | | | |
| Endothelial | +⁸ | +⁹ | +⁸ | +² | − | | +⁸ | +⁸ | +⁸ | +⁸ |
| dysfunction* | | | | | | | | | | |
| Dyslipidemia | +¹⁰ | +¹¹ | +¹⁰ | +⁴ | +¹² | +⁸ | +¹³ | | +¹⁴ | +¹⁵ |
| Inflammation | +¹⁶ | +¹⁷ | +¹⁸ | +⁴ | +‡⁴ | +⁸ | +¹⁹ | +²⁰ | | +²¹ |
| Insulin Resistance/ | +¹⁰ | +¹¹ | | +²² | +²³ | +⁸ | +²⁴ | +¹⁵ | +¹⁸ | |
| diabetes | | | | | | | | | | |
| Other Risk Factors | | | | | | | | | | |
| Aging | +¹⁰ | +¹¹ | +²⁵ | +²⁶ | +²⁷ | +⁸ | +²⁸ | +²⁹ | +³⁰ | +³¹ |
| Obesity | +¹⁰ | +³² | +³³ | +³⁴ | +³⁵ | +⁸ | +³³ | +³³ | +³⁶ | +³³ |
| Unexercised State | +¹⁰ | +³⁷ | +³⁸ | +³⁹ | +⁴⁰ | +⁸ | +⁴¹ | +⁴² | +⁴² | +⁴³ |
| Stress | +⁴⁴ | +⁴⁵ | +⁴⁶ | +⁴⁷ | +⁴⁸ | +⁴⁴ | +⁴⁴ | +⁴ | +⁴ | +⁴⁹ |
| ↑ Blood Pressure | +⁴⁴ | +⁴⁵ | +⁴⁶ | +⁴⁷ | +⁴⁸ | +⁴⁴ | +⁴⁴ | +⁴ | +⁴ | +⁴⁹ |
| Hypertension | | | | | | | | | | |
| Smoking | +¹⁰ | +¹¹ | +⁵⁰ | +⁵¹ | +‡⁵¹ | +⁸ | +⁵¹ | +⁵² | +¹⁸ | +⁵³ |
| Circadian Rhythm | +⁵⁴ | +⁵⁴ | +§⁵⁵ | +⁵⁴ | +‡⁵⁴ | +‡⁵⁴ | +⁵⁴ | +⁵⁶ | +⁵⁷ | +§⁵⁵ |
| Cold | +⁵⁸ | +⁵⁹ | +⁶⁰ | +⁶¹ | +‡⁶¹ | +‡⁶¹ | +⁶¹ | +⁶² | +⁶³ | +⁶⁴ |

*Expresses a tendency toward thrombosis and vasoconstriction
†↑ glucose levels only for sympathetic activation
‡By inference, as opposite agent causes opposite effect
§Plasma glucose levels
− No Information Found

Table 2 summarizes the beneficial effects of pharmaceutical agents on CV related diseases, on risk factors for adverse CV events, and on other situations not directly related to CV events. The superscripts in the table reference the publications, listed at the end of the specification, in which the effects of pharmaceutical agents on adverse CV related diseases have been reported for each particular entry.

TABLE 2
Beneficial Effects of Pharmaceutical Agents on Diseases,
on Parasympathetic Activation, and on Other Situations

| Pharmaceutical Agents | | | | |
|---|---|---|---|---|
| | | Anti- | | Estrogen/ |
| | | Angio- | PPAR* | Proges- |
| Statins | Aspirin | tension | agonists | terone |

Diseases

| | | | | | |
|---|---|---|---|---|---|
| Prevent IHD | +¹⁰ | +†⁶⁵ | +⁶⁶ | +⁶⁷ | cr⁶⁸ |
| Treat IHD | +⁶⁹ | +⁷⁰ | +⁶⁶ | +⁷¹ | − |
| Prevent Hypertension | +§⁷² | − | +‡⁷³ | +⁷⁴ | +⁷⁵ |
| Treat Hypertension | +§⁷⁶ | +§†⁷⁷ | +¹¹ | − | − |
| Prevent Diabetes | +⁷⁸ | − | +⁷⁹ | +⁸⁰ | +⁸¹ |
| Treat Diabetes | +⁸² | − | +⁸³ | +⁸⁴ | − |

Parasympathetic Activation and Improvement of
Sympathetic activation, i.e. Major Risk Factors

| | | | | | |
|---|---|---|---|---|---|
| ↑ Parasympath. | +¹² | +⁸⁵ | +⁸⁶ | − | +⁸⁷ |
| Activity | | | | | |
| ↓ Sympathetic | +⁸⁸ | − | +⁷⁹ | +⁸⁹ | +⁹⁰ |
| activity | | | | | |
| ↓ Endothelial | +⁸ | +⁹¹ | +⁸ | +⁹² | +⁸ |
| dysfunction | | | | | |
| ↓ Thrombosis | +⁹³ | +†⁹⁴ | +⁹⁵ | +⁹² | x⁹⁶ |
| ↓ Dyslipidemia | +¹⁰ | +⁹⁷ | +⁹⁸ | +⁹⁹ | +¹⁰⁰ |
| ↓ Inflammation | +¹⁰¹ | +†⁹⁴ | +¹⁰² | +⁹² | +¹⁰³ |
| ↓ Insulin | +⁸² | +⁹⁷ | +⁷⁹ | +⁸⁴ | +¹⁰⁴ |
| Resistance | | | | | |
| Other Situations/ | | | | | |
| Disorders | | | | | |
| ↑ Cognitive | +¹⁰⁵ | +¹⁰⁶ | +¹⁰⁷ | − | +¹⁰⁸ |
| function | | | | | |
| ↑ Bone density | +¹⁰⁹ | +¶^{¶110} | − | cr¹¹¹ | +¹¹² |
| ↓ Alzheimer's | +cr¹¹³ | +†¹¹⁴ | +¹¹⁴ | +‡¹¹⁵ | +¹¹⁶ |
| disease | | | | | |
| ↓ Atrial | +¹¹⁷ | − | +¹¹⁸ | − | − |
| fibrillation | | | | | |
| ↓ Cancer | +¹¹⁹ | +¹²⁰ | +¹²¹ | cr¹²² | x¹¹² |

+Positive association
crConflicting results
− No information found
naNo association
xNegative association
*Peroxisome proliferator-activated receptor
†Low dose aspirin
‡Proposed
§Known to lower blood pressure
¶By inference, as opposite agent causes opposite action
#Possibility raised
**Further studies needed
††Study of fructose-fed rats
§§Study of growing rabbits
¶¶Plus COX-2 inhibitor

Table 3 summarizes the beneficial effects of certain “lifestyle agents”, for example healthy living habits (e.g., maintaining proper weight, exercise, stress reduction, and healthy dietary habits) on CV related diseases, on risk factors for adverse CV events, and on other situations not directly related to adverse CV events. The superscripts in the table reference the publications, listed at the end of the specification, in which the effects of lifestyle agents on CV related diseases have been reported for each particular entry.

TABLE 3
Beneficial Effects of Lifestyle Agents on Diseases, on Parasympathetic Activation, and on Other Situations

| | Lifestyle Agents | | | | | | | |
|---|---|---|---|---|---|---|---|---|
| | | | | Moderate | Fish oil/ | | Mediterrean | Diet high in |
| | Exercise | ↓ Weight | ↓ Stress | alcohol | Fish | Nuts | diet | flavonoids |
| Diseases | | | | | | | | |
| Prevent IHD | +¹²³ | +¹⁰ | +⁴⁴ | +¹²⁴ | +¹²⁵ | +¹²⁶ | +¹²⁷ | +¹²⁸ |
| Treat IHD | +¹²³ | − | +⁴⁴ | +¹²⁴ | +¹²⁹ | − | +¹²⁷ | − |
| Prevent Hypertension | +¹³⁰ | +¹³⁰ | +¶¹³¹ | x¹³⁰ | +¹²⁵ | − | +§¹³² | +¹³³ |
| Treat Hypertension | +¹³⁰ | +¹³⁰ | +¹³⁴ | x¹³⁰ | +¹³⁵ | − | +§¹³² | − |
| Prevent Diabetes | +⁴² | +¹³⁶ | +¶#¹³⁷ | +¹³⁸ | − | +¹³⁹ | +¹⁴⁰ | − |
| Treat Diabetes | +⁴² | +¹³⁶ | +¶¹⁴¹ | +**¹³⁸ | − | − | − | − |
| Parasympathetic Activation | | | | | | | | |
| and Improvement of | | | | | | | | |
| Sympa thetic activation, | | | | | | | | |
| i.e. Major Risk Factors | | | | | | | | |
| ↑ Parasympath. Activity | +¹⁴² | +¹⁴³ | +¹⁴⁴ | x¹⁴⁵ | +¹⁴⁶ | − | − | − |
| ↓ Sympathetic activity | +³⁹ | +¹⁴⁷ | +¹⁴⁸ | x¹⁴⁹ | +¹⁵⁰ | − | − | − |
| ↓ Endothelia dysfunction | +¹⁵¹ | +¹⁵² | +¶¹⁵³ | +¹⁵⁴ | +¹⁴⁶ | +¹²⁶ | +¹⁵⁵ | +¹²⁸ |
| ↓ Thrombosis | +⁴¹ | +⁴¹ | +¶⁴¹ | +¹⁵⁶ | +¹⁴⁶ | − | − | +¹⁵⁷ |
| ↓ Dyslipidemia | +⁴² | +¹⁵⁸ | +¹³⁴ | +¹⁵⁹ | +¹⁴⁶ | +¹⁶⁰ | na¹²⁷ | +¹⁶¹ |
| ↓ Inflammation | +¹⁶² | +¹⁵² | +¹⁶³ | +¹⁶⁴ | +¹⁴⁶ | − | +¹⁵⁵ | +¹²⁸ |
| ↓ Insulin Resistance | +⁴² | +¹⁵⁸ | +¶¹⁶⁵ | +¹⁰⁴ | cr¹⁶⁶ | +**¹⁶⁷ | +¹⁵⁵ | na††¹³³ |
| Other Situations/Disorders | | | | | | | | |
| ↑ Cognitive function | +¹⁶⁸ | x¹⁶⁹ | +¶¹⁰⁸ | +¹⁷⁰ | +¹⁷¹ | − | − | +¹⁷² |
| ↑ Bone density | +¹⁷³ | x¹⁷⁴ | − | +¹⁷⁵ | x§§¹⁷⁶ | − | − | +¹⁶¹ |
| ↓ Alzheimer's disease | +¹⁷⁷ | − | +¶¹⁷⁸ | +¹⁷⁹ | +¹⁸⁰ | − | +¹⁸¹ | +‡¹⁸² |
| ↓ Atrial fibrillation | − | − | +¶¹⁸³ | x¹⁸⁴ | +¹⁸⁵ | − | − | − |
| ↓ Cancer | +¹⁸⁶ | +¶¹⁸⁷ | +¶¹⁸⁸ | x¹⁸⁹ | − | − | +‡¹⁹⁰ | +‡¹⁹¹ |

+Positive association
crConflicting results
− No information found
naNo association
xNegative association
*Peroxisome proliferator-activated receptor
†Low dose aspirin
‡Proposed
§Known to lower blood pressure
¶By inference, as opposite agent causes opposite action
#Possibility raised
**Further studies needed
††Study of fructose-fed rats
§§Study of growing rabbits
¶¶Plus COX-2 inhibitor

As evidenced by the data represented in Tables 2 and 3, preventative factors (i.e., pharmaceutical and lifestyle agents) are considered to operate by a single opposing mechanism involving parasympathetic activation. Multiple major and diverse preventative agents for IHD, hypertension, and diabetes exhibit findings which are opposite those of risk factors for IHD, hypertension, and diabetes, and this information also provides evidence that preventative agents operate by a single mechanism.

Evidence for parasympathetic activation by preventative factors is based on findings that multiple and diverse pharmaceutical and lifestyle agents express a decrease in sympathetic activity and an increase in parasympathetic activity; also, findings of preventative factors are directly opposite those of sympathetic fight/fight and risk factors.

While Tables 2 and 3 are not designed to provide full evidence for the idea that any preventative factor can oppose any risk factor, the risk factors of endothelial dysfunction, a tendency to thrombosis, dyslipidemia, inflammation, and insulin resistance are improved by multiple pharmaceutical and lifestyle agents. However, the findings that pharmaceutical and lifestyle preventative agents rather uniformly express parasympathetic activation and attributes of parasympathetic activation provides strong evidence that preventative factors operate by one mechanism—and thus would oppose and completely or partially counteract any risk factor, including the risk attributable to one or more first pharmaceutical agents, for example, a nonselective or COX-2 selective NSAID. As additional evidence that pharmaceutical and lifestyle preventative agents operate by one mechanism, the various agents generally were shown to have the same actions of improving cognitive function and bone density, and decrease the incidence of Alzheimer's disease, artrial fibrillation, and cancer.

Thus, based on a synthesis of the data from the literature, which is uniquely reflected in Tables 1-3, there is unexpected close similarity of findings of multiple and diverse risk factors, and multiple and diverse preventative factors; these similarities are considered to offer strong evidence that risk and preventative factors operate by a single opposing mechanism and through a risk/prevention balance.

As an NSAID, for example, basically represents a risk factor, the principle of overbalancing risk factors by added preventative factors should improve the risk/prevention balance and allow for significant reduction of the risk of CV events from administration of non-selective NSAIDs and/or COX-2 inhibitors. As the risk of an adverse CV event induced by one or more of a first pharmaceutical agent is identified with specific mediators of homeostasis, it should be useful to discuss these mediators.

For example, it is generally accepted that the risk of adverse CV events from COX-2 inhibitors is due to a thromboxane/prostacyclin imbalance. Because the risk of COX-2 inhibitor induced infarction ordinarily is couched only in terms of the balance between thromboxane A₂ and prostacyclin, this frame of reference does not allow consideration of additive preventative measures. From the stance of the altered homeostatic theory, the important issue is the overall balance of risk vs. preventative factors, and this overall balance involves more than thromboxane A₂ and prostacyclin. For myocardial infarction, one critical issue is the overall balance between thrombosis/vasoconstriction and anti thrombosis/vasodilation. Limiting the discussion only to endothelium there are at least six pro-thrombotic, five vasoconstricting, six anti-thrombotic, and six vasodilating substances released from endothelium (Verma S, Anderson T J. Circulation. 2002; 105:546-549).

Without wishing to be bound by any particular theory, there are likely two different types of risk factors that lead to adverse CV events. First are risk factors that cause general sympathetic activation, with concomitant expression of the four attributes of sympathetic activation, i.e., endothelial dysfunction expressing thrombosis/vasoconstriction, inflammation, dyslipidemia, and insulin resistance. Risk factors that lead to general sympathetic activation include, for example, emotional stress and cold exposure. Second are risk factors that activate only one of the four sympathetic attributes but lead to a general sympathetic activation of the autonomic nervous system through the expression of the other three attributes. One sympathetic attribute influences the others because of the highly integrated nature of the autonomic nervous system. Examples of the second type of risk factor are diet-induced dyslipidemia and infection-induced inflammation. It can be seen in Table 1 that dyslipidemia and inflammation are associated with sympathetic activation and lead to the activation of the other three corresponding attributes of sympathetic activation.

For CV events, the critical factor is the expression of thrombosis/vasoconstriction by risk factors; CV events generally are attributed to thromboses, and has been attributed to spasm (Hellstrom Med Hypotheses 2003; 60:36-51). Because of this, the adverse CV risk of COX-2 inhibitors is considered to be due to an overbalance of thrombosis/vasoconstriction, which can be opposed by the anti-thrombosis/vasodilative forces of the preventative measures of the invention.

From available information, it appears that COX-2 inhibitors directly cause a heightened tendency toward thrombosis/vasoconstriction because of an imbalance of thromboxane and prostacyclin. This imbalance would favor CV events, as CV events generally are attributed to thrombosis, and also have been attributed to vasoconstriction (Hellstrom H R. Med Hypotheses 2003; 60:36-51). As standard preventative measures express anti thrombosis/vasodilation, preventative measures would oppose a COX-2 inhibitor-derived tendency towards thrombosis/vasoconstriction. The presence of a COX-2 inhibitor-induced tendency toward thrombosis/vasoconstriction should tend to “pull” the sympathetic nervous system towards sympathetic activation and expression of the other attributes of sympathetic activation, i.e., inflammation, dyslipidemia, and insulin resistance.

While the standard position appears to be that the class effect of COX-2 inhibitors is to favor CV events, COX-2 inhibitors can be considered in a different light, which is considered to offer an explanation for their sometimes disparate effects. It is suggested that the class effect of COX-2 inhibitors is to shift homeostasis beneficially toward parasympathetic dominance—which includes improvement of endothelial dysfunction (which expresses thrombosis/vasoconstriction), dyslipidemia, inflammation, and insulin resistance. COX-2 inhibitors are anti-inflammatory (Antman E M, et al. Circulation 2005; 112:759-70) and NSAIDs improve other components of an adverse sympathetic shift; nonselective and selective NSAIDs improve hyperlipidemia (Kourounakis A P, et al. Exp Mol Pathol 2002; 73:135), and celecoxib improves insulin resistance (González-Ortiz M, et al. Horm Metabol Res 2001; 33:250) and endothelial dysfunction (Widlansky M E, et al. Hypertension 2003; 42:310).

The tendency of COX-2 inhibitors to cause CV events is regarded as a side effect due to drug-induced expression of thrombosis/vasoconstriction. Whether any particular COX-2 inhibitor induced CV events is considered to be dependent on whether side effects of thrombosis/vasoconstriction overbalances class effects of anti-thrombosis/vasodilation. The worsening by parecoxib of endothelial dysfunction in individuals with essential hypertension (Bulut D J et al. Hypertens 2003; 21:1663) is regarded as evidence of dominance of side effects over class effects.

While the balance between class effects and side effects offers a method of explaining the complex findings of COX-2 inhibitors, the critical aspect of the invention is the opposition of thrombosis/vasoconstriction of risk factors by anti-thrombosis/vasodilation of preventative factors. Accordingly, not all pharmaceutical agents which favor adverse CV events are considered to operate through a class vs. side effect mechanism. For example, sympathomimetic agents which favor adverse CV events likely operate through their basic tendency to favor thrombosis/vasoconstriction.

To reduce risk of CV events, the altered homeostatic theory underlying the present invention assumes that adding preventative measures would upregulate available anti-thrombotic and vasodilative agents to oppose the risk of thromboxane A₂. As examples of how beneficial pharmaceutical agents might specifically reduce the risk of infarction, statins increase bioavailability of nitric oxide (anti-thrombotic and vasodilatory), and anti-angiotensin agents promote nitric oxide accumulation and levels of bradykinin (anti-thrombotic and vasodilative).

Many individuals have multiple risk factors, and preventative agents used to counteract, for example, NSAID-induced imbalance of thromboxane and its consequent tendency toward thrombosis/vasoconstriction should also reduce the impact of other risk factors. All of the risk factors associated with adverse CV events are considered to generate thrombosis/vasoconstrictive forces, and anti-thrombosis/vasodilatory forces from preventative agents should act against other risk factors. Risk factors are considered to act additively, and anti-thrombosis/vasodilatory forces from preventative factors should act additively against the totality of thrombosis/vasoconstrictive forces from risk factors—NSAID-induced and otherwise.

Statins and anti-angiotensin agents are examples of added pharmaceutical preventative agents which should significantly reduce the adverse CV risk of, for example, COX-2 inhibitors. Statins have a proven ability to prevent IHD, and anti-angiotensin agents, also known to prevent infarction, should be especially useful to counteract the increase in blood pressure and destabilization of hypertension control by, for example, COX-2 inhibitors. In individuals with a more significant risk, multiple pharmaceutical agents, for example, a statin and or more pharmaceutical agents from a group which includes anti-angiotensin agents, vasodilators as calcium channel blockers, PPAR agents, fibric acid derivates, inhibitors of intestinal cholesterol, beta blockers, folic acid, and thiazide can be used. Also, anti-inflammatory actions from a preventative agents as statins, ACE inhibitors, have been postulated to help stabilize atherosclerosis plaques (Ambrose J A Circulation 2002; 105:2000-2004), and thus should act to reduce the incidence of plaque rupture and risk of infarction.

The preventative agents of the present invention, (e.g., pharmaceutical agents for reducing the risk of CV events and healthy living habits) will also help stabilize atherosclerotic coronary artery plaques. It generally is accepted that plaque rupture directly induces infarction (Fuster et al. N Engl J Med 1992; 326:242-250, 310-318), and that lipid lowering by diet and statins reduce the incidence of infarction by stabilizing plaques by, for example, cholesterol reduction and reduction of inflammation (Brown et al. Circulation. 1993; 87:1781-1791; Libby et al. Am J Med 1998; 104:14s⁻¹⁸S; Ambrose et al. Circulation 2002; 105:2002-2004). Plaque material is highly thrombogenic (Fernändez-Ortiz et al. J Am Coll Cardiol 1994; 23:1562-1569), and whether thromboses are primary or secondary, prevention of plaque rupture should reduce the incidence of adverse CV events. While plaque rupture generally is not discussed in context to, for example, NSAID-induced infarctions, pharmaceutical and lifestyle preventative agents used in the invention should operate to stabilize plaques as they reduce lipid levels and reduce inflammation. Consistent with this position, it recently was proposed that in addition to statins, agents such as angiotensin converting enzyme inhibitors should help stabilize plaques (Ambrose et al. Circulation 2002; 105:2000-2004).

It is likely that the risk of infarction by, for example, COX-2 inhibitors will be lower in individuals with a significantly low general risk of IHD—as healthy, non obese, well exercised young adults. In such individuals, the total weight of preventative measures should reduce the risk of COX-2 inhibitors. And using the lowest effective dose would help maintain a favorable risk/prevention balance in such individuals.

In one embodiment of the present invention, the methods and compositions, and programs herein are directed at reducing the risk of adverse CV events associated with one or more first pharmaceutical agents, particularly an NSAID and especially a COX-2 inhibitor, in patients who are in need of therapy with one or more first pharmaceutical agents, where the risk of adverse CV event comprises the potential for cardiac arrest, acute or chronic myocardial infarction, coronary heart disease, ischemia, stroke, peripheral vascular disease, claudication, worsening angina, restenosis, stroke, atherosclerosis, sudden cardiac death and/or cardiac arrhythmias.

A patient in need of NSAID therapy may require the medication for the treatment of pain, especially post-traumatic pain and pain associated with inflammation, or may have an inflammation-related medical condition(s) including, but not limited to, arthritis (such as rheumatoid arthritis and osteoarthritis) and other autoimmune disorders (e.g., multiple sclerosis, myasthenia gravis, Alzheimer's disease, glomerulonephritis, Crohn's disease, Guillain-Barre Syndrome, lupus erythematosus and irritable bowel syndrome); atherosclerosis; asthma and other lung disorders, including respiratory distress syndrome; skin conditions and injuries such as psoriasis, bullous pemphigoid, lichen planas, burns and wounds; sepsis and other infections; hemorrhagic shock; and multiple organ system failure. Such conditions also include medical procedures such as organ transplantation (e.g., lung), tissue grafts, hemodialysis, and cardiopulmonary bypass surgery, where recovery may be inhibited or delayed as a result of inflammation.

In a particular embodiment of the present invention, the methods and compositions, and programs herein are directed at reducing the risk of adverse CV events associated with COX-2 inhibitors in patients who are in need of COX-2 inhibitor therapy. In addition to the treatments described above for NSAIDs, COX-2 inhibitor therapy has also been used in the treatment of certain cancers, especially prostate cancer (U.S. Pat. No. 6,534,540), colonic polyps, and in the treatment of autosomal dominant polycystic kidney disease (U.S. Publication No. 2004/0024042).

In another embodiment of the present invention, the methods and compositions, and programs of this invention are directed at healthy individuals who are in need of therapy with a first pharmaceutical agent, particularly NSAID and COX-2 inhibitor therapy, and who are not at an elevated CV risk due to any other risk or disease factor prior to the initiation of therapy with a first pharmaceutical agent.

In yet another embodiment of the present invention, the methods and compositions, and programs of this invention are directed at individuals who are in need of therapy with one or more first pharmaceutical agents, particularly NSAID and COX-2 inhibitor therapy, and who are at an elevated CV risk prior to the initiation of therapy with a first pharmaceutical agent, where the individuals who are at elevated CV risk include, but are not limited to, those with systemic lupus erythematosus; diabetes; angina pectoris; manifest coronary artery disease; stroke; hypertension; hypercholesterolemia; kidney disease; Chlamydia infection; Bartonella infection; obstructive pulmonary disease; who are on hemodialysis; who have received an organ transplant; who are obese; who are elderly; who have a family history of heart disease, atherosclerosis, or stroke; who are or have been cigarette smokers; or who have a history of myocardial infarction, transient ischemic attacks, stroke, atherosclerosis, stress, dyslipidemia, endothelial dysfunction, or peripheral vascular disease.

In another embodiment, the methods compositions, and programs of the invention include first pharmaceutical agents other than NSAIDs that increase the incidence of adverse CV events because of their specific chemistry. No matter the exact mechanism which a drug causes an increase in adverse CV events, the mechanism is considered to involve increasing the tendency toward thrombosis/vasoconstriction. Thus, preventative agents which express anti thrombosis/vasodilation should reduce the incidence of adverse CV events associated with these other first pharmaceutical agents. These agents include, but are not limited to, SERMSs, such as raloxifene, muraglitazar, sympathomimetic agents, for example anti-ADHD agents, and acetaminophen.

Recognition that the pharmaceutical agent raloxifene increases the risk of stroke (Barrett-Conner E. et al. N. Engl. J. Med. 2006; 355:125-37) provides evidence that SERMs can increase adverse CV events while decreasing the risk of osteoperosis and the risk of invasive breast cancer in women.

Recognition that the pharmaceutical agent muraglitazar increases the risk of myocardial infarct and stroke (Nissen S E, et al. JAMA. 2005; 294:2581-2586) provides evidence that other drugs can also increase adverse CV events even though they reside in a class of pharmaceuticals predicted to reduce CV risk. Muraglitazar is a PPAR agent, whose class action should reduce the incidence of infarction (Table 2). Consistent with a protective class effect, muraglitazar is a dual α and γ PPAR agent, a type which increases insulin sensitivity and improves dyslipidemia. However, similar to COX-2 inhibitors, it appears that side effects of muraglitazar overbalance these protective class effects.

As noted above, the methods and compositions, and programs herein are also directed at reducing the risk of adverse CV events associated with sympathomimetic agents, including anti-ADHD pharmaceuticals. The FDA has recently recommended a “black box” warning describing the risks of adverse CV events associated with stimulant-based ADHD medications (Nissen, S. E. NEJM 2006; 354:1445). Pharmaceutical agents used in the treatment of ADHD include, but are not limited to combinations of amphetamine aspartate, amphetamine sulfate, dextroamphetamine saccharate, and dextroamphetamine sulfate (Adderall® and Adderall XR®), methylphenidate (Concerta®, Ritalin LA®, Ritalin SR®, Methalyn CD®, and Metadate®), dextroamphetamine (Dexedrine®), and atomoxetine (Strattera®).

Furthermore, the methods and compositions, and programs herein are also directed at reducing the risk of adverse CV events associated with acetaminophen. Recent studies have shown that use of acetaminophen at high frequency or dose is associated with a significant increase in the risk of adverse CV events (Circulation 2006; 113:1578-1587).

According to the prevention invention, preventative measures act against the first pharmaceutical agent and also against standard risk factors; in each case, the risk/prevention balance is improved.

### NSAIDs

As used herein, the terms “nonsteroidal anti-inflammatory drug” and “NSAID” are synonymous and refer to non-opioid analgesics characterized in that they are non-steroidal drugs which act as anti-inflammatory, analgesic and antipyretic agents. This class of drugs is well known in the art, see, for example, Chapter 27 of Goodman, L. and Gilman, A. (“The Pharmacological Basis of Therapeutics”, 9th edition, Pergamon press, New York, 1996). Within this class of drugs are salicylates, such as aspirin; pyrazolone derivatives such as phenylbutazone, onyphenbutazone, antipyrine, aminopyrine, dipyrone, metamizol, phenazone, propyphenazone and apazone; indomethacin; sulindac; fenamates, such as mefenamic, meclofenamic, flufenamic, tolfenamic and etofenamic acids; COX-2 inhibitors such as meloxicam, piroxicam, celecoxib, valdecoxib and rofecoxib; aryl acetic acid and propionic acid compounds such as ibuprofen, suprofen, oxprozin, carprofen, fenoprofen, fenoprofen calcium; naproxen; indoprofen; ketoprofen; flurbiprofen and tolmetin. Also included within NSAIDs are compounds within the class including zomepirac sodium; piroxicam, tenoxicam, diflunisal or proquazone; phenylacetic acid derivatives such as alclofenac, diclofenac, etodolac, ketorolac tromethamine, toradol; and nabumetone.

It should be noted that, although the FDA has stated that the risk of CV events appears to be a class effect for all COX-2 and non-selective NSAIDs, the NSAID aspirin has been associated with cardioprotective effects. For an account of aspirin's ability to provide cardioprotective effects including the reduction of the incidence of myocardial infarction, see, Schieffer et al. Am. J. Cardiol. 91:12H-18H (2003). Therefore, in a specific embodiment of the present invention, the term NSAID excludes aspirin.

### Selective COX-2 Inhibitors

As used herein, the terms “COX-2 inhibitor” (sometimes “selective COX-2 inhibitor”) and “COX-2 selective NSAID” are synonymous and refer to NSAIDs that specifically inhibit COX-2 and which have little or no effect on COX-1. For example, at a dosage that caused a 50% inhibition of COX-2, a COX-2 inhibitor would inhibit COX-1 by less than 10%.

All pharmaceutically acceptable COX-2 inhibitors are included in this invention, for example, those disclosed in U.S. Pat. Nos. 5,393,790; 5,409,944; 5,418,254; 5,420,343; 5,436,265; 5,474,995; 5,476,944; 5,486,534; 5,510,368; 5,521,213; 5,536,752; 5,547,975; 5,550,142; 5,552,422; 5,565,482; 5,576,339; 5,580,985; 5,585,504; 5,593,994 and 5,596,008. Specific COX-2 inhibitors suitable for the practice of the invention include, but are not limited to, dimethyl furanone, ON-09300, ON-09250, rofecoxib, XU-745, RWJ-63556, GR-253035, L-768277, TJN-120P, FR-123826, DFP, SC-57666, NS-398, darbufelone, flosulide, nabumetone (oral), Nobex, S-33516, S-2474, tilmacoxib, SC-58451, L-746483, L-748731, L-752860, PGV-20229, PD-098120-0003, FR-188582, BMS-347070, UP-45421, SC-XX906, deracoxib, L-784512, FR-140423, L-748780, CS-179, L-745337, L-761066, ABT-963, CS-502, L-778736, BIRL-790, PD-138387, PD-164387, L-783003, L-758115, SC-58231, SC-58236, APF-328, Ro-26-2198, DRF-4848, tricyclic dextrocannabinoids, acanthoic acid, PMI-006, TRPV 1 agonists, MM-5312079, PMI-005, OSU-03012, OCID-2065, UR-14048, NO-naproxen, JB-7/G, AF-3161, LAU-0501, LM-4108, L-776967, triterpenoid iNOS/COX-2 inhibitors, M-5011, NO-enhanced NSAIDs, PH-686464, nitroflurbiprofen, p38 kinase inhibitors, 644784, SVT-2016, LR-3001, ajulemic acid, IDEA-070, nitroflurbiprofen, PMI-001, P-54, cimicoxib, CS-706, LAS-34475, GW-406381, celecoxib, parecoxib, meloxicam, diclofenac, nimesulide, etoricoxib, lumiracoxib, and valdecoxib.

Compounds that selectivity inhibit the activity of COX-2 can be readily identified by using assays well known in the art. An international group of scientists published a consensus review related to COX-2 screening assays in: Brooks et al; Rheumatology 1999; 38: 779-788. In this consensus paper, the committee stated that the Human Whole Blood Assay developed by Patrignani et al (J Pharmacol Exp Ther 1994; 271: 1705-12) was the best assay available for assessing inhibition of COX-1 and COX-2, or evaluating new COX inhibitors. More recently, the William Harvey Modified Human Whole Blood Assay was developed as an extension of the original whole blood assay, and most of the NSAID drugs, as well as the newer COX-2 inhibitors have been screened using this method.

In a specific embodiment, the COX-2 inhibitors for use in the practice of the present invention are celecoxib (CELEBREX®); rofecoxib (VIOXX®); valdecoxib (BEXTRA®); meloxicam; etoricoxib; lumiracoxib; tiracoxib; cimicoxib; and piroxicam; deracoxib; etodolac; and dermaxx.

In a more particular embodiment, the COX-2 inhibitors are celecoxib, rofecoxib and valdecoxib.

As indicated above, the present invention involves co-administering NSAIDs, especially COX-2 inhibitors, with one or more pharmaceutical agents for reducing the risk of CV events including, but not limited to, ACE inhibitors and/or ARBs to reduce the risk of or prevent adverse CV events caused by administration of NSAIDs. In the context of the present invention, the pharmaceutical agent for reducing the risk of CV events may be administered with an NSAID alone, or in combination with other pharmaceutical agent for reducing the risk of CV events. Examples of those agents which may thus be co-administered with the NSAIDs are described below.

### Hydroxymethylglutaryl-Coenzyme A (HMG-CoA) Reductase Inhibitors (“Statins”)

HMG-CoA (3-hydroxy-3-methylglutaryl-coenzyme A) reductase is the enzyme which catalyzes the rate limiting step of cholesterol biosynthesis. HMG-CoA reductase inhibitors, also known as statins, are molecules which inhibit the enzymatic activity of HMG-CoA reductase and have been used to treat patients suffering from hypercholesterolemia. The first such inhibitor (compactin or Mevastatin) was isolated in 1976 (Endo, A. et al. F.E.B.S. Lett., 72: 323-326, 1976) and since then many other natural and chemically modified versions of Mevastatin have been identified and developed for clinical use.

Currently available statins include lovastatin, simvastatin, pravastatin, fluvastatin, cerivastatin and atorvastatin. Lovastatin (disclosed in U.S. Pat. No. 4,231,938) and simvastatin (ZOCOR®; disclosed in U.S. Pat. No. 4,444,784 and WO 00/53566) are administered in the lactone form. After absorption, the lactone ring is opened in the liver by chemical or enzymatic hydrolysis, and the active hydroxy acid is generated. Pravastatin (PRAVACHOL®; disclosed in U.S. Pat. No. 4,346,227) is administered as the sodium salt. Fluvastatin (LESCOL®; disclosed in U.S. Pat. No. 4,739,073), atorvastatin calcium salt (LIPITOR®; see U.S. Pat. No. 5,273,995) and cerivastatin sodium salt (also known as rivastatin; see U.S. Pat. No. 5,177,080) are also well known statins.

Recent studies have shown that, in addition to treatment of hyperlipidemia, HMG-CoA reductase inhibitors are useful in the treatment of acne and/or skin aging (see, e.g. Breton, L. et al. U.S. Pat. No. 5,902,805); can increase nitric oxide (NO)-mediated vasodilation and blood vessel relaxation (see e.g., Liao, J. K. et al. WO 99/18952); and can help prevent a second or additional myocardial infarction (see, e.g., Behounek, B. D. et al. U.S. Pat. No. 5,674,893; Olukotun, A. Y. et al. U.S. Pat. No. 5,622,985).

Behounek, B. D. et al. (U.S. Pat. No. 5,674,893) have shown that patients with one or more risk factors for a coronary event such as hypercholesterolemia, who are treated with an HMG-CoA reductase inhibitor, such as pravastatin, experience a rapid marked and significant reduction in adverse CV events. Thus, although a certain number of patients having one or more risk factors for coronary events are expected to suffer an adverse CV event, such as a myocardial infarction and/or unstable angina, it has unexpectedly been found that such patients when treated with an HMG-CoA reductase inhibitor, such as pravastatin, have a rapid and sizable reduction in such adverse CV events. What is even more remarkable is the fact that such reduction in adverse CV events occur within one year and usually within 6 months of treatment and even sooner. This is especially significant inasmuch as until now it has been the generally held view that a treatment effect on cardiac event rates appears only after a lag phase of 2 years, as seen in the Coronary Primary Prevention Trial (JAMA 1984; 251:351-364) and the Helsinki Heart Study (N. Engl. J. Med. 1987; 317:1237-1245).

According to the PROspective Study of Pravastatin in the Elderly at Risk (PROSPER) study, the HMG Co-A reductase inhibitor pravastatin was shown to reduce the risk of CV events (15% reduction versus placebo) in those with existing (secondary prevention) and in those at high risk of developing (primary prevention) vascular disease (Shepherd, J et al. The Lancet 360:1623 (2002)).

The terms “cholesterol-lowering agent”, “cholesterol-lowering drug” and “lipid-lowering agent” as used herein refer to a pharmacologically active, pharmaceutically acceptable agent that, when administered to a human subject, has the effect of modifying serum cholesterol levels. More particularly, the cholesterol-lowering agent lowers serum low density lipoprotein (LDL) cholesterol levels, or inhibits oxidation of LDL cholesterol, whereas high density lipoprotein (HDL) serum cholesterol levels may be lowered, remain the same, or be increased.

As used herein, the terms “hydroxymethylglutaryl coenzyme A (HMG-CoA) reductase inhibitors” and “statins” are synonymous and refer to members of a class of compounds that inhibit 3-hydroxy-3-methylglutaryl coenzyme A (HMG-CoA) reductase. The HMG-CoA reductase inhibitors belong to the broader class of lipid lowering agents. This enzyme catalyzes the conversion of HMG-CoA to mevalonate, which is an early and rate-limiting step in the biosynthesis of cholesterol. Examples of HMG-CoA reductase inhibitors that may be used include but are not limited to lovastatin (MEVACOR®; see U.S. Pat. No. 4,231,938), simvastatin (ZOCOR®; see U.S. Pat. No. 4,444,784), pravastatin (PRAVACHOL®; see U.S. Pat. No. 4,346,227), fluvastatin (LESCOL®; see U.S. Pat. No. 5,354,772), atorvastatin (LIPITOR®; see U.S. Pat. No. 5,273,995), cerivastatin (also called rivastatin; see U.S. Pat. No. 5,177,080), mevastatin (see U.S. Pat. No. 3,883,140), fluindostatin (Sandoz XU-62-320), velostatin (also called synvinolin; see U.S. Pat. Nos. 4,448,784 and 4,450,171), and compounds related to these as described in the cited references. Some other examples of HMG-CoA reductase inhibitors that may be used are, without limitation, presented in U.S. Pat. No. 6,264,938 at Table 1 and U.S. Pat. No. 5,622,985, columns 3 through 6. All pharmaceutically acceptable HMG-CoA reductase inhibitors are included in this invention.

In a specific embodiment, the statins of the present invention are atorvastatin, fluvastatin, lovastatin, pravastatin, rosuvastatin, and simvastatin, and especially atorvastatin.

Compounds that inhibit the activity of HMG-CoA reductase can be readily identified by using assays well known in the art; see, as examples, the assays described or cited in U.S. Pat. No. 4,231,938 at column 6, and in International Patent Publication WO 84/02131 at pp. 30-33.

### The Renin-Angiotensin System (RAS) and the Anti-Angiotensins

The renin-angiotensin system (RAS) is an endocrine cascade which traditionally has been thought to regulate blood volume, vascular tone, and Na homeostasis. Angiotensin-converting enzyme (ACE), the final enzyme in the cascade, is the key catalytic step in production of the peptide hormone angiotensin II (Ang II). The enzyme cleaves two amino acids from the inactive prohormone angiotensin I (Ang I) to form the biologically active octapeptide Ang II, a potent vasoconstrictor. In addition, ACE inactivates bradykinin (BK) in a two-step cleavage process first to the inactive peptide BK 1-7 followed by further cleavage to BK 1-5 (Stewart, Handbook of Inflammation, Volume 6: Mediators of the Inflammatory Process, pp. 189-217, Elsevier Science Publishers (1989)). ACE is also capable of hydrolyzing other peptides, including some neuropeptides such as gonadotropin-releasing hormone (Skigdel and Erdos, J. Clin. and Exper. Theory and Practice 19987; A9(2&3):243-259).

The RAS has recently been implicated as an important adverse pathogenic mechanism in CV disease (Dzau, Circulation 1988; 77:4-13). As stated above, ACE plays a key role in the RAS. The enzyme converts Ang I to Ang II and also hydrolyzes vasodilator and antiproliferative kinins such as BK. The Ang II produced by the action of ACE also has powerful non-vasoconstrictive effects. Ang II can promote proliferation of myocardial and vascular smooth muscle and cause neointimal hyperplasia after arterial wall injury (Lindpaintner et al. J. Cardiovasc. Pharmacol. 1992; 20:S41-S47). Although Ang I can be converted to Ang II in the absence of ACE (Kinoshita et al. J. Biol. Chem. 1991; 266:19192-19197, Urata et al. J. Clin. Invest. 1993; 91:1269-1281, Urata et al. Circ. Res. 1990; 66:883-890), administration of an ACE inhibitor substantially diminishes the proliferative and pressor effects of Ang I, suggesting that ACE-mediated production of Ang II is an important physiologic process (Swales and Dzau, Am. Heart J. 1992; 123:1412-1413). The RAS has been implicated in CV disease primarily because of its role in fluid volume and blood pressure control, but the growth promoting effects of Ang II on smooth muscle and myocardium may also be directly involved in disease processes. In addition, increased ACE-mediated hydrolysis of BK, a potent local vasodilator, may have adverse effects. The RAS has also been implicated in left ventricular remodeling after myocardial infarction. This results in progressive left ventricular dilation and contractile dysfunction (J. Cardiovasc. Pharmacol. 1992; 20:S41-S47)

The terms “anti-angiotensin agent” and “inhibitor of the renin-angiotensin system” as used herein are synonymous and refer to a pharmacologically active, pharmaceutically acceptable agent that inhibits, directly or indirectly, the adverse effects of angiotensin, particularly angiotensin II. Included, without limitation, are agents that: inhibit angiotensin II synthesis; inhibit angiotensin II binding to the AT₁ receptor; or inhibit renin activity.

### Angiotensin-Converting Enzyme (ACE) Inhibitors

ACE inhibitors have major roles as vasodilators in hypertension and CHF and are among the most efficient drugs for treating these disorders (see, e.g., Opie et al. “Angiotensin Converting Enzyme Inhibitors and Conventional Vasodilators,” in Lionel H. Opie, Drugs for the Heart, Third Edition, WB Saunders Company, 1991, p106). Several clinical trials indicate that ACE inhibitors prolong survival in a broad spectrum of patients with myocardial infarction and heart failure, ranging from those who are asymptomatic with ventricular dysfunction to those who have symptomatic heart failure but are normotensive and hemodynamically stable. For example, one study demonstrated a 40% reduction in mortality at 6 months in patients with severe heart failure (The CONSENSUS Trial Study Group, N. Engl. J. Med. 1987; 316:1429; The CONSENSUS Trial Study Group, N. Engl. J. Med. 1991; 325:293).

The benefits of treatment are not restricted to survival. The addition of an ACE inhibitor to diuretic therapy improves the control of heart failure, an important symptomatic benefit. This reduces the need for hospitalization and probably improves the patient's quality of life. There may also be economic benefits for the health care system. Since their introduction in the mid-1980s, angiotensin converting enzyme (ACE) inhibitors have become well established for the treatment of hypertension and heart failure.

Selection of the patients to be treated is not based on the presence or absence of altered ACE levels or the presence of any of the polymorphisms in the gene, however, but solely on the observation of symptoms in which the known vasodilator properties of the ACE inhibitors have been proven to be useful. These patients are typically treated with relatively low doses of the ACE inhibitors in an amount effective to decrease blood pressure.

Recent studies have shown that the use of ACE inhibitors in patients with myocardial infarction has improved survival and reduced the rates of non-fatal CV events, especially when these agents are used for long term treatment in high risk patients such as those with signs of heart failure, evidence of left ventricular systolic dysfunction, or both. (Pfeffer, M A. et al. New Engl. J. Med. 2003; 349:1893)

The Heart Outcomes Prevention Evaluation (HOPE) study investigated whether the addition of the ACE inhibitor ramipril to the current medical regimen of high-risk patients with diabetes mellitus could lower risks of adverse CV events. The rate of the combined primary outcome of myocardial infarction, stroke, and cardiovascular death was significantly lower in the ramipril group compared to the placebo group (relative risk reduction was 25%) (See, The Lancet 2000; 355:253).

The term “angiotensin converting enzyme (ACE) inhibitors” as used herein refers to any compound that inhibits the conversion of angiotensin I to angiotensin II. Because angiotensin I has only about 1% of the hypertensive activity of angiotensin II, ACE inhibitors are generally effective in reducing blood pressure and the other adverse CV effects caused by angiotensin II. ACE has numerous substrates other than angiotensin I, including bradykinin. By interfering with the conversion of bradykinin, ACE inhibitors increase bradykinin levels; this mechanism may contribute to the efficacy of ACE inhibitors.

Numerous ACE inhibitors have been synthesized. Most of these compounds can be classified into three groups based on their chemical structure: (1) sulfhydryl-(also called mercapto-) containing ACE inhibitors, including captopril and agents that are structurally related to captopril, such as fentiapril, pivalopril, zofenopril and alacepril; (2) dicarboxyl-containing ACE inhibitors, including enalapril and agents that are structurally related to enalapril, such as lisinopril, benazepril, quinapril, moexipril, ramipril, spirapril, perindopril, indolapril, pentopril, indalapril and cilazapril; and (3) phosphorus-containing ACE inhibitors, structurally related to fosinopril. Many of the ACE inhibitors are esters developed for high oral bioavailability, but with low potency in themselves; they must be converted to particular metabolites in the body that have potent activity.

ACE inhibitors are well known in the art, and the use of any pharmaceutically acceptable ACE inhibitor, including any of those mentioned in the preceding paragraph, is included in this invention, including mixtures thereof and/or their pharmaceutically acceptable salts. Some further examples of ACE inhibitors that may be used in the practice of this invention are, without limitation, AB-103, ancovenin, benazeprilat, BRL-36378, BW-A575C, CGS13928C, CL242817, CV-5975, Equaten, EU-4865, EU-4867, EU-5476, foroxymithine, FPL 66564, FR-900456, Hoe-065, 15B2, indolapril, ketomethylureas, KR1-1177, KR1-1230, L681176, libenzapril, MCD, MDL-27088, MDL-27467A, moveltipril, MS-41, nicotianamine, pentopril, phenacein, pivopril, rentiapril, RG-5975, RG-6134, RG-6207, RGH0399, ROO-911, RS-10085-197, RS-2039, RS 5139, RS-86127, RU-44403, S-8308, SA-291, spiraprilat, SQ26900, SQ-28084, SQ-28370, SQ-28940, SQ-31440, Synecor, utibapril, WF-10129, Wy-44221, Wy-44655, Y23785, Yissum, P-0154, zabicipril, Asahi Brewery AB-47, alatriopril, BMS 182657, Asahi Chemical C-11, Asahi Chemical C-112, Dainippon DU-1777, mixanpril, Prentyl, zofenoprilat, 1 (-(1-carboxy-6-(4-piperidinyl) hexyl) amino)-1-oxopropyl octahydro-1H-indole-2-carboxylic acid, Bioproject BP1.137, Chiesi CHF 1514, Fisons FPL-66564, idrapril, perindoprilat, Servier S-5590, alacepril, benazepril, captopril, cilazapril, delapril, enalapril, enalaprilat, fosinopril, fosinoprilat, imidapril, lisinopril, perindopril, quinapril, ramipril, ramiprilat, saralasin acetate, temocapril, trandolapril, trandolaprilat, ceranapril, moexipril, quinaprilat, spirapril, and those listed in U.S. Pat. No. 6,248,729.

In a specific embodiment, the ACE inhibitors are benazepril, captopril, cilazapril, delapril, enalapril, fentiapril, fosinopril, indolapril, lisinopril, moexipril, perindopril, pivopril, quinapril, ramipril, spirapril, trandolapril, and zofenopril; especially captopril, enalapril, fosinopril, lisinopril, quinapril, ramipril, and trandolapril. An exemplary ACE inhibitor is ramipril.

### Angiotensin II Receptor Blockers (ARBs)

Alternative metabolic pathways of angiotensin production and the side effects of ACE inhibitors led to a search for an alternative method of blocking angiotensin II. Since all known pressor effects of angiotensin II are mediated through the AT₁ receptor, a medication that blocked these receptors would theoretically share therapeutic effects of the ACE inhibitors. This new approach yielded the angiotensin II receptor blockers (ARBs). The first blockade of the angiotensin II receptors was achieved in the 1970s with saralasin acetate, which had a short duration and had to be given parenterally. Losartan, the first orally administered angiotensin II type 1 (AT₁) receptor blocker, became available in 1995.

ARBs lower blood pressure by interfering with the action of angiotensin II, blockading the AT₁ receptor site. This obstructs the action of angiotensin II regardless of its site or mechanism of production.

Because the ARBs and the ACE inhibitors each interrupt the renin-angiotensin system (RAS) there has been a tendency to see these two classes as alternatives to one another. The ACE inhibitors, however, do not fully prevent conversion of angiotensin I to angiotensin II during chronic treatment, probably because enzymes other than the ACE may take a greater role in facilitating conversion when ACE is blocked. In general, clinical trials comparing the antihypertensive efficacy of the two classes have shown comparable blood pressure-lowering effects.

The potential for the ARBs to treat patients with CHF has been determined by two Evaluation of Losartan in the Elderly (ELITE) studies. The first of these studies compared Losartan (50 mg) with the ACE inhibitor captopril (50 mg) three times daily in patients with heart failure and appeared to show a significant reduction in mortality for patients on the ARB (vs. ACE inhibitor). The second study (ELITE II) showed no significant difference in the clinical endpoints between the two treatments, but did demonstrate that losartan was better tolerated (vs. captopril). Recently, the FDA approved the ARB valsartan for the treatment of heart failure in patients who cannot tolerate ACE inhibitors.

ARBs as a class of agents have made a minor contribution to the treatment of hypertension. These agents effectively reduce blood pressure and are well tolerated. Other clinical trials have focused, however, on a much wider use of ARBs in conditions such as congestive heart failure (CHF), postmyocardial infarction management, and diabetic neuropathy. Recent studies have provided evidence that ARBs might confer target organ protection in hypertension that is equal to, and possibly better than, the benefits provided by the more conventional antihypertensive agents (e.g., ACE inhibitors). Therefore, ARBs have been, and continue to be, carefully scrutinized for their ability to prevent CV events and stroke.

Recently ARBs have shown efficacy in the reduction of CV events as demonstrated in the VALsartan In Acute myocardial Infarction Trial (VALIANT) (Nickenig G. Circulation 110:1013 (2004). Pfeffer et al. demonstrated that valsartan had improved survival and reduced the rates of non-fatal CV events in patients with myocardial infarction (See, New Engl. J. Med. 2003; 349:1893). At a target dose of 160 mg twice daily, valsartan was proven to be as effective as the ACE inhibitor captopril as a proven regimen for in improving survival and reducing cardiovascular morbidity.

The terms “angiotensin II receptor antagonists”, “angiotensin II antagonists” and “angiotensin II receptor blockers (ARBs)” are synonymous and refer to any agent that inhibits the binding of angiotensin II to its known receptors. Angiotensin II binds to angiotensin subtype I (AT₁) and subtype 2 (AT₂) receptors, as well as to several other receptors. All the known physiological effects of angiotensin II are apparently due to its binding to, and activation of, the AT₁ receptor, which is abundantly expressed in the tissues affected by angiotensin II. AT₂ receptor is common in some fetal tissues but is scarce in adult tissues; to date, no known function has been discovered for it. Many orally active, nonpeptide angiotensin II receptor antagonists have been developed. Most of these are directed at the AT₁ receptor, but due to concerns about unbalanced activation of the AT₂ receptor, some newer angiotensin II receptor antagonists target both AT₁ and AT₂ receptors. Angiotensin II receptor antagonists are generally highly specific, having very little effect on other hormone receptors or ion channels.

Any pharmaceutically active antagonists of the AT₁ angiotensin II receptor may be used in this invention. Some examples of angiotensin II receptor antagonists suitable for use herein are saralasin (including saralasin acetate), candesartan (including candesartan cilexetil), CGP-63170, EMD-66397, KT3-671, LRB/081, valsartan, A-81282, BIBR-363, BIBS-222, BMS-184698, CV11194, EXP-3174, KW-3433, L-161177, L-162154, LR-B/057, LY-235656, PD150304, U-96849, U-97018, UP-275-22, WAY-126227, WK-1492.2K, YM-31472, losartan (including losartan potassium), E-4177, EMD-73495, eprosartan, HN-65021, irbesartan, L-159282, ME-3221, SL-91.0102, tasosartan, telmisartan, UP-269-6, YM-358, CGP-49870, GA-0056, L-159689, L-162234, L-162441, L-163007, PD-123177, A81988, BMS-180560, CGP-38560A, CGP-48369, DA-2079, DE-3489, DuP-167, EXP-063, EXP-6155, EXP-6803, EXP-7711, EXP-9270, FK-739, HR-720, ICI D6888, IC1-D7155, IC1-D8731, isoteoline, KR1-1177, L-158809, L-158978, L-159874, LR B087, LY-285434, LY-302289, LY-315995, RG-13647, RWJ-38970, RWJ-46458, S-8307, S-8308, saprisartan, sarmesin, WK-1360, X-6803, ZD-6888, ZD-7155, ZD-8731, BIBS39, CI-996, DMP-811, DuP-532, EXP-929, L163017, LY-301875, XH-148, XR-510, zolasartan, and PD-123319.

In a specific embodiment, the angiotensin II receptor antagonists include losartan (which is the prototype and best known angiotensin II receptor antagonist), irbesartan, eprosartan, canadesartan, valsartan, olmesartan, telmisartan, zolasartin, and tasosartan. A particularly exemplary ACE inhibitor is losartan.

### Healthy Living Habits

In an alternative embodiment of the present invention is a total program for reducing the risk of adverse CV events associated with the administration of NSAIDs that involves (1) the co-administration of an NSAID with one or more pharmaceutical agents for reducing the risk of adverse CV events; and/or (2) following a recommendation to begin healthier living habits. The term “healthy living habits” and “lifestyle agents” as used herein are synonymous and include any healthy living habit that can be recommended by a physician for the purpose of reducing the risk of adverse CV events. Healthy living habits suitable for the practice of this invention include, but are not limited to, implementing an exercise program, loss of weight, stress reduction, imbibition of moderate levels of alcohol, implementing a polymeal diet and/or a diet high in, for example, fish oil or fish, nuts, and/or flavinoids. The recommendation can be, for example, a physician's recommendation or a recommendation included in the pharmaceutical agent's or package insert

Based on projections on efficacy of multiple drugs and lifestyle changes in preventing infarcts, it is possible that a program based on multiple pharmaceutical preventative agents and proper lifestyles will likely reduce the incidence of drug-induced infarction significantly. It has been estimated that multiple drugs would reduce the incidence non-drug induced infarction by up to 80% (Wald N J, Law M R BMJ 2003; 326: 1419), a proper diet by up to 75% (Franco O H, et al. BMJ 2004; 329:1147), and multiple drugs plus lifestyle changes over 90% (Robinson J G, Maheshwari N. Am J Cardiol 2005; 95:373).

### Salts, Prodrugs, and Metabolites

Any of the foregoing active agents may be administered in the form of a salt, ester, amide, prodrug, active metabolite or the like, provided that the salt, ester, amide, prodrug, or active metabolite is pharmaceutically acceptable and pharmacologically active in the present context. Salts, esters, amides, prodrugs or metabolites of the active agents may be prepared using standard procedures known to those skilled in the art of synthetic organic chemistry and described, for example, by J. March, Advanced Organic Chemistry: Reactions, Mechanisms and Structure, 4th Edition (New York: Wiley-Interscience, 1992).

For example, acid addition salts are prepared from a drug in the form of a free base using conventional methodology involving reaction of the free base with an acid. Suitable acids for preparing acid addition salts include both organic acids, e.g., acetic acid, propionic acid, glycolic acid, pyruvic acid, oxalic acid, malic acid, malonic acid, succinic acid, maleic acid, fumaric acid, tartaric acid, citric acid, benzoic acid, cinnamic acid, mandelic acid, methanesulfonic acid, ethanesulfonic acid, p-toluenesulfonic acid, salicylic acid, and the like, as well as inorganic acids, e.g., hydrochloric acid, hydrobromic acid, sulfuric acid, nitric acid, phosphoric acid, and the like. An acid addition salt may be reconverted to the free base by treatment with a suitable base. Conversely, preparation of basic salts of acid moieties that may be present on an active agent may be carried out in a similar manner using a pharmaceutically acceptable base such as sodium hydroxide, potassium hydroxide, ammonium hydroxide, calcium hydroxide, trimethylamine, or the like. Preparation of esters involves transformation of a carboxylic acid group via a conventional esterification reaction involving nucleophilic attack of an RO⁻ moiety at the carbonyl carbon. Esterification may also be carried out by reaction of a hydroxyl group with an esterification reagent such as an acid chloride. Esters can be reconverted to the free acids, if desired, by using conventional hydrogenolysis or hydrolysis procedures. Amides may be prepared from esters, using suitable amine reactants, or they may be prepared from an anhydride or an acid chloride by reaction with ammonia or a lower alkyl amine. Prodrugs and active metabolites may also be prepared using techniques known to those skilled in the art or described in the pertinent literature. Prodrugs are typically prepared by covalent attachment of a moiety that results in a compound that is therapeutically inactive until modified by an individual's metabolic system.

Other derivatives and analogs of the active agents may be prepared using standard techniques known to those skilled in the art of synthetic organic chemistry, or may be deduced by reference to the pertinent literature. In addition, chiral active agents may be in isomerically pure form, or they may be administered as a racemic mixture of isomers.

### Formulations and Routes of Administration

The methods and compositions discussed above are compatible with any dosage form or route of administration. Thus, agents may be administered orally, intranasally, rectally, sublingually, buccally, parenterally, or transdermally. Dosage forms may include tablets, trochees, capsules, caplets, dragees, lozenges, parenterals, liquids, powders, and formulations designed for implantation or administration to the surface of the skin. In general, it is expected that oral dosage forms will be the most convenient. All dosage forms may be prepared using methods that are standard in the art (see e.g., Remington's Pharmaceutical Sciences, 16th ed. A. Oslo. ed., Easton, Pa. (1980), and later editions).

Active ingredients may be used in conjunction with any of the vehicles and excipients commonly employed in pharmaceutical compositions, e.g., talc, gum arabic, lactose, starch, magnesium stearate, cocoa butter, aqueous or non-aqueous solvents, oils, paraffin derivatives, glycols, etc. Coloring and flavoring agents may also be added to preparations designed for oral administration. Solutions can be prepared using water or physiologically compatible organic solvents such as ethanol, 1-2 propylene glycol, polyglycols, dimethyl sulfoxide, fatty alcohols, triglycerides, partial esters of glycerin, and the like. Parenteral compositions containing active ingredients may be prepared using conventional techniques and include sterile isotonic saline, water, 1,3-butanediol, ethanol, 1,2-propylene glycol, polyglycols mixed with water, Ringer's solution, etc.

In a specific embodiment, the present invention provides for a pharmaceutical composition, wherein the cyclooxygenase-2 inhibitor and one or more of the foregoing pharmaceutical agents for reducing or prevent the risk of CV events are in a oral dosage form.

Oral dosage forms are used to administer the combination of active agents, and include tablets, capsules, caplets, solutions, suspensions, and/or syrups, and may also comprise a plurality of granules, beads, powders, or pellets that may or may not be encapsulated. Such dosage forms are prepared using conventional methods known to those in the field of pharmaceutical formulation and described in the pertinent texts, e.g., in Gennaro, A. R., editor, Remington: The Science and Practice of Pharmacy, 20th Edition (Lippincott, Williams and Wilkins, 2000). Tablets and capsules represent the most convenient oral dosage forms, in which cases solid pharmaceutical carriers are employed.

Tablets may be manufactured using standard tablet processing procedures and equipment. One method for forming tablets is by direct compression of a powdered, crystalline, or granular composition containing the active agent(s), alone or in combination with one or more carriers, additives, or the like. As an alternative to direct compression, tablets can be prepared using wet-granulation or dry-granulation processes. Tablets may also be molded rather than compressed, starting with a moist or otherwise tractable material; however, compression and granulation techniques are particularly useful.

In addition to the active agent(s), then, tablets prepared for oral administration using the method of the invention will generally contain other materials such as binders, diluents, lubricants, disintegrants, fillers, stabilizers, surfactants, coloring agents, and the like.

Binders are used to impart cohesive qualities to a tablet, and thus ensure that the tablet remains intact after compression. Suitable binder materials include, but are not limited to, starch (including corn starch and pregelatinized starch), gelatin, sugars (including sucrose, glucose, dextrose and lactose), polyethylene glycol, waxes, and natural and synthetic gums, e.g., acacia sodium alginate, polyvinylpyrrolidone, cellulosic polymers (including hydroxypropyl cellulose, hydroxypropyl methylcellulose, methyl cellulose, ethyl cellulose, hydroxyethyl cellulose, and the like), and Veegum.

Diluents are typically necessary to increase bulk so that a practical size tablet is ultimately provided. Suitable diluents include dicalcium phosphate, calcium sulfate, lactose, cellulose, kaolin, mannitol, sodium chloride, dry starch, and powdered sugar. Lubricants are used to facilitate tablet manufacture; examples of suitable lubricants include, for example, magnesium stearate, calcium stearate, and stearic acid. Stearates, if present, usually represent no more than approximately 2 wt. % of the drug-containing core. Disintegrants are used to facilitate disintegration of the tablet, and are generally starches, clays, celluloses, algins, gums, or crosslinked polymers.

Fillers include, for example, materials such as silicon dioxide, titanium dioxide, alumina, talc, kaolin, powdered cellulose, and microcrystalline cellulose, as well as soluble materials such as mannitol, urea, sucrose, lactose, dextrose, sodium chloride, and sorbitol.

Stabilizers are used to inhibit or retard drug decomposition reactions that include, by way of example, oxidative reactions. Surfactants may be anionic, cationic, amphoteric, or nonionic surface active agents.

The dosage form may also be a capsule, in which case the active agent-containing composition may be encapsulated in the form of a liquid or solid (including particulates such as granules, beads, powders, or pellets). Suitable capsules may be either hard or soft, and are generally made of gelatin, starch, or a cellulosic material, with gelatin capsules most common. Two-piece hard gelatin capsules are usually sealed, such as with gelatin bands or the like. See, for example, Remington: The Science and Practice of Pharmacy, cited supra, which describes materials and methods for preparing encapsulated pharmaceuticals. If the active agent-containing composition is present within the capsule in liquid form, a liquid carrier is necessary to dissolve the active agent(s). The carrier must be compatible with the capsule material and all components of the pharmaceutical composition, and must be suitable for ingestion.

In a particular embodiment of the present invention, the oral dosage form is a unit dosage form.

When two or more active agents are combined in a single pharmaceutical dosage form, possible interactions among the active agents, and among the active agents and the excipients, must be considered. Such consideration is well within the purview of those skilled in the art of pharmaceutical formulation. For example, an acidic agent may react with basic compounds or alkali esters in such a way as to cause hydrolysis or the degradation of one of more of the active agents. When necessary, the present composition thus encompasses pharmaceutical compositions wherein two or more of the active agents are separated from each other within the pharmaceutical dosage form, by, for example, separating potentially interacting compounds from each other within the pharmaceutical dosage form, as in separate flat layers of a tablet (e.g., a bilayer or trilayer tablet), concentric layers, coated beads or granules (which may be incorporated into a compressed tablet or into a capsule), and/or by using buffers (see, for example, U.S. Pat. No. 6,235,311). It will also be appreciated by those in the art that such dosage forms, wherein two or more active agents are physically separated from the other active agents, can be manufactured so that different active agents will have different release profiles, e.g., if one active agent is formulated with an enteric coating, another active agent is formulated in a sustained release matrix, and the like. Alternatively, non-reactive pharmaceutically active derivatives of one or more of the potentially interacting compounds may be used.

Solid dosage forms, whether tablets, capsules, caplets, or particulates, may, if desired, be coated so as to provide for delayed release. Dosage forms with delayed release coatings may be manufactured using standard coating procedures and equipment. Such procedures are known to those skilled in the art and described in the pertinent texts, e.g., in Remington, supra. Generally, after preparation of the solid dosage form, a delayed release coating composition is applied using a coating pan, an airless spray technique, fluidized bed coating equipment, or the like. Delayed release coating compositions comprise a polymeric material, e.g., cellulose butyrate phthalate, cellulose hydrogen phthalate, cellulose proprionate phthalate, polyvinyl acetate phthalate, cellulose acetate phthalate, cellulose acetate trimellitate, hydroxypropyl methylcellulose phthalate, hydroxypropyl methylcellulose acetate, dioxypropyl methylcellulose succinate, carboxymethyl ethylcellulose, hydroxypropyl methylcellulose acetate succinate, polymers and copolymers formed from acrylic acid, methacrylic acid, and/or esters thereof.

Sustained release dosage forms provide for drug release over an extended time period, and may or may not be delayed release. Generally, as will be appreciated by those of ordinary skill in the art, sustained release dosage forms are formulated by dispersing a drug within a matrix of a gradually bioerodible (hydrolyzable) material such as an insoluble plastic, a hydrophilic polymer, or a fatty compound, or by coating a solid, drug-containing dosage form with such a material. Insoluble plastic matrices may be comprised of, for example, polyvinyl chloride or polyethylene. Hydrophilic polymers useful for providing a sustained release coating or matrix cellulosic polymers include, without limitation: cellulosic polymers such as hydroxypropyl cellulose, hydroxyethyl cellulose, hydroxypropyl methyl cellulose, methyl cellulose, ethyl cellulose, cellulose acetate, cellulose acetate phthalate, cellulose acetate trimellitate, hydroxypropylmethyl cellulose phthalate, hydroxypropylcellulose phthalate, cellulose hexahydrophthalate, cellulose acetate hexahydrophthalate, and carboxymethylcellulose sodium; acrylic acid polymers and copolymers, e.g., formed from acrylic acid, methacrylic acid, acrylic acid alkyl esters, methacrylic acid alkyl esters, and the like, e.g., copolymers of acrylic acid, methacrylic acid, methyl acrylate, ethyl acrylate, methyl methacrylate and/or ethyl methacrylate, with a terpolymer of ethyl acrylate, methyl methacrylate, and trimethylammonioethyl methacrylate chloride (sold under the tradename Eudragit RS); vinyl polymers and copolymers such as polyvinyl pyrrolidone, polyvinyl acetate, polyvinylacetate phthalate, vinylacetate crotonic acid copolymer, and ethylene-vinyl acetate copolymers; zein; and shellac, ammoniated shellac, shellac-acetyl alcohol, and shellac n-butyl stearate. Fatty compounds for use as a sustained release matrix material include, but are not limited to, waxes generally (e.g., carnauba wax) and glyceryl tristearate.

### Therapeutic Use

The NSAIDs, especially the COX-2 inhibitors, are particularly useful in the treatment of pain, e.g., pain due to migraine headache, and inflammation. Thus, the invention includes methods of treating these conditions by administering an agent for reducing the risk of adverse CV events in combination with an NSAID, and especially a COX-2 inhibitor. These agents should be given in a co-timely manner and should be delivered in an amount sufficient to reduce pain or inflammation. In general, it is expected that the drugs will be given within 24 hours of one another.

In a particular embodiment, the active agents be administered in a unit dosage form, as emphasized above. However, in some cases, a patient may be given each active agent in its own separate dosage form, or a combination of individual “combination” dosage forms containing two or more of the present active agents. When separate dosage forms are used, the NSAID or COX-2 inhibitor, HMG-CoA reductase inhibitor, ACE inhibitor, and/or ARB can be administered at essentially the same time (concurrently), or at separately staggered times (sequentially). Optimum beneficial effects are achieved when the active blood level concentrations of each active agent are maintained at substantially the same time, meaning that simultaneous drug administration is generally preferred. Other dosing schedules, such as administering the NSAID once per day and administering the HMG-CoA reductase, twice, or more times per day, are also contemplated. A single oral dosage form comprising all the active agents is, however, more convenient for the patient and the healthcare provider. Such a dosage form provides convenience and simplicity for the patient, thus increasing the chances for patient compliance, especially in patients who already take multiple medications due to existing heart disease or other diseases.

### Dosages

With respect to therapeutic agents, it is expected that the skilled practitioner will adjust dosages on a case by case basis using methods well established in clinical medicine. Nevertheless, the following general guidelines with respect to the NSAIDs, COX-2 inhibitors, statins, ACE inhibitors, and ARBs may be of help.

With regard to the COX-2 inhibtors, a satisfactory result may be obtained in dosages employed, for example, for rofecoxib, celecoxib, and valdecoxib as indicated in the Physician's Desk Reference, such as in an amount within the range of from about 5 to 800 mg, per day in single or divided doses, and particularly from about 10 to about 400 mg per day in single or divided doses. For all other COX-2 inhibitors, the dosages of the present invention are from about 1 to about 1000 mg and more particularly from about S to 500 mg.

Celecoxib (CELEBREX®) is particularly useful when contained in tablets of from about 100 to 200 mg. Recommended dosages are typically 100 mg twice per day or 200 mg once per day (see, Bolten, J., Rheumatolog. Suppl., 1998; 51:2-7) but can be used at a dosage of up to 800 mg daily. The recommended daily dose for osteoarthritis is 200 mg/day, and for acute pain is 400 mg initial dose and then 200 mg bid.

Rofecoxib (VIOXX®) for oral administration, manufactured in tablets of 12.5, 25 or 50 mg and in an oral suspension containing either 12.5 mg or 25 mg rofecoxib per 5 ml. The recommended initial daily dosage for the management of acute pain is typically 50 mg, but can be used at a dosage of up to 200 mg daily. The recommended daily dose for osteoarthritis is typically 12.5 mg/day, and for rheumatoid arthritis is typically 25 mg/day.

Valdecoxib (BEXTRA®) for oral administration, manufactured in tablets of 10 or 20 mg. The recommended daily dose for osteoarthritis is typically 10 mg/day, and for dysmenorrhea is typically 20 mg twice a day (bid), but can be used at a dosage of up to 200 mg daily.

With regard to the statins, a satisfactory result may be obtained employing the HMG-CoA reductase inhibitor in dosages employed, for example, for pravastatin, lovastatin and simvastatin as indicated in the Physician's Desk Reference (PDR), such as in an amount within the range of from about 1 to 2000 mg, per day in single or divided doses, and more particularly from about 4 to about 200 mg per day in single or divided doses.

In one embodiment the dosage form of the invention comprises: an HMG-CoA reductase inhibitor in an amount of from about 0.5 to about 100 mg, and more particularly from about 5 to about 80 mg.

In a further embodiment, the dosage form of the invention comprises: approximately 10 mg to approximately 80 mg, and more particularly approximately 25 mg to approximately 60 mg, of an HMG-CoA reductase inhibitor selected from the group consisting of atorvastatin, fluvastatin, lovastatin, pravastatin, rosuvastatin, and simvastatin.

According to the PDR, fluvastatin (LESCOL®) is usually prescribed in unit dosages of 20 and 40 mg with 80 mg extended release dosages available. Lovastatin (ALTOCOR®, Mevacor is usually prescribed in unit dosages of 10, 20 and 40 mg with extended release 10, 20, 40 and 60 mg. Pravastatin (PRAVACHOL®) is usually prescribed in unit dosages of 10, 20, 40 and 80 mg. Simvastatin (ZOCOR®) is usually prescribed in unit dosages of 5, 10, 20, 40, and 80 mg. Atorvastatin (LIPITOR®) is usually prescribed in unit dosages of 10, 20, 40 and 80 mg. Rosuvastatin (CRESTOR®) is usually prescribed in unit dosages of 5, 10, 20 and 40 mg.

For the practice of the present invention, it is especially advantageous to use as low dose of a statin as possible, as side effects are reported to be dose related (Grundy S M. et al. Circulation. 2005; 111:3016-3019). Equivalent doses of the various statins can be determined by the method of Grundy et al. (See Table 1 of: Grundy S M et al. Circulation 2004; 110; 227:239). The effectiveness of a statin in the lowering of cholesterol can be used as measure of its effectiveness for reducing the risk of CV events associated with the administration of COX-2 inhibitors. Thus, an exemplary dosage regimen would be the administration of atorvastatin (LIPITOR®) at a dosage of 5 to 20 mg daily as it has shown the greatest reduction in LDL (39% reduction with a 10 mg dose) as compared to other statins.

In the case of ACE inhibitors, specific dosages are, for example, from about 1 mg to about 800 mg, more particularly from about 5 mg to about 600 mg, and especially from about 10 mg to about 400 mg.

For example, preferred dosages according to the PDR are, for example, from about 5 mg to about 20 mg, preferably 5 mg, 10 mg or 20 mg of benazepril; from about 6.5 mg to 100 mg, preferably 6.25 mg, 12.5 mg, 25 mg, 50 mg, 75 mg or 100 mg of captopril; from about 2.5 mg to about 20 mg, preferably 2.5 mg, 5 mg, 10 mg or 20 mg of enalapril; from about 10 mg to about 20 mg, preferably 10 mg or 20 mg of fosinopril; from about 2.5 mg to about 4 mg, preferably 2 mg or 4 mg of perindopril; from about 5 mg to about 20 mg, preferably 5 mg, 10 mg or 20 mg of quinapril; or from about 1.25 mg to about 5 mg, preferably 1.25 mg, 2.5 mg, or 5 mg of ramipril.

The daily dosage may be provided in either a single dose or multiple dose regimen, with the latter being generally preferred. These are simply guidelines since the actual dose must be carefully selected and titrated by the attending physician based upon clinical factors unique to each patient. The optimal daily dose will be determined by methods known in the art and will be influenced by factors such as the age of the patient, the disease state, side effects associated with the particular agent being administered and other clinically relevant factors. In some cases, a patient may already be taking medications at the time that treatment with the present combination is initiated. These other medications may be continued provided that no unacceptable adverse side effects are reported by the patient.

Since two or more active agents are being used together in a combination therapy, the potency of each of the agents and the interactive effects achieved by combining them together must also be taken into account. A consideration of these factors is well within the purview of the ordinarily skilled clinician for the purpose of determining the therapeutically effective or prophylactically effective dosage amounts. For example, a combination of a COX-2 inhibitor with a lower dose of statins/ACE inhibitors/ARBs is envisioned for individuals with a lower risk of infarction (i.e., individuals whose only major risk factor is the administration of a COX-2 inhibitor and who are otherwise healthy). A combination of a COX-2 inhibitor with a higher dose of statins/ACE inhibitors/ARBs is envisioned for individuals with a higher risk of infarction (i.e., individuals who have CV risk factors in addition to those arising from the administration of a COX-2 inhibitor).

In the case of ARBs, specific dosages are, for example, from about 1 mg to about 800 mg, particularly from about 2 mg to about 600 mg, and more particularly from about 2 mg to about 300 mg.

For example, specific dosages according to the PDR are, for example, from about 2 mg to about 32 mg, particularly 4 mg, 8 mg or 16 mg and 32 mg of candaesatan; from about 200 mg to about 600 mg, particularly 400 mg or 600 mg of eprosartan; from about 75 mg to about 300 mg, particularly 150 or 300 mg of irbesartan; from about 12.5 mg to about 100 mg, particularly 25 mg, 50 mg, or 100 mg of losartan; from about 2.5 mg to about 40 mg, particularly 5 mg, 20 mg, or 40 mg of olmesartan; from about 10 mg to about 80 mg, particularly 20 mg, 40 mg or 80 mg of telmisartan; or from about 20 mg to about 320 mg, particularly 30 mg, 80 mg, 160 mg, or 320 mg of valsartan.

In a further embodiment of the present invention is a dosage regimen for individuals who are at elevated risk of seriously adverse CV events and who are already taking a statin, ACE inhibitor, or ARB (prior to the administration of an NSAID and especially a COX-2 inhibitor) to reduce this risk. A consideration of these factors is well within the purview of the ordinarily skilled clinician. For example, upon administration of an NSAID, the clinician can determine an effective dose of any additional or supplemental dosage of a statin, ACE inhibitor, or ARB that may be necessary. Alternatively, if a patient is already on a regimen that includes a statin and there is a risk of side effects from an increase of statin dosage, a clinician may decide to co-administer the COX-2 inhibitor with an effective dosage of an ACE inhibitor or an ARB.

The risk/prevention balance of the present invention provides a more satisfactory solution to, for example the COX-2 inhibitor problem, than the current solution, which is based essentially on minimizing risk by reducing or eliminating these agents (For the current solution, See Antmann E M, DeMets, D, Loscalzo, J Circulation 2005; 112:759-770). With the addition of counterbalancing preventative agents, CV-favoring pharmaceutical agents should be able to be prescribed at appropriate dosing levels and duration to reduce risk of adverse CV events. However, due caution should be taken in individuals at high risk for adverse CV events who already are taking standard preventative measures. In appropriate cases, the principle of overbalancing risk allows increasing preventative forces by higher doses of preventative pharmaceutical agents as statins and/or adding more preventative agents, such as, additional pharmaceutical agents and healthy living habits.

Without further elaboration, it is believed that one skilled in the art can, using the preceding description, utilize the present invention to its fullest extent. The following examples are, therefore, to be construed as merely illustrative, and not limitative of the remainder of the disclosure in any way whatsoever.

### EXAMPLE 1

### Daily Dosage Regimen for Co-administration of the COX-2 Inhibitor Celecoxib (CELEBREX®) with the Statin Atorvastatin (LIPITOR®)

Table 4 provides dosage regimens for the co-administration of celecoxib with atorvastatin. As illustrated in the Table 4, the ratio of CELEBREX® to LIPITOR® is adjusted based on the patient's need for effective pain relief and the patient's risk of a seriously adverse CV event (e.g., myocardial infarction). For example, a patient who has a high risk of infarct prior to COX-2 inhibitor therapy is co-administered CELEBREX®/LIPITOR® in a ratio of 10:1. A patient who has a low risk of infarct prior to COX-2 inhibitor therapy is co-administered CELEBREX®/LIPITOR® in a ratio of 20:1.

TABLE 4

| | Moderate dose of Celebrex | High dose of Celebrex |
|---|---|---|
| Lower risk | Celebrex 200 mg/Lipitor | Celebrex 400 mg/Lipitor |
| of infarct | 10 mg | 20 mg |
| Higher risk | Celebrex 200 mg/Lipitor | Celebrex 400 mg/Lipitor |
| of infarct | 20 mg | 40 mg |

### EXAMPLE 2

### Daily Dosage Regimen for Co-Administration of the COX-2 Inhibitor Rofecoxib (VIOXX®) with the Statin Atorvastatin (LIPITOR®)

Table 5 provides dosage regimens for the co-administration of rofecoxib with atorvastatin. As illustrated in the Table 4, the ratio of VIOXX® to LIPITOR® is adjusted based on the patient's need for effective pain relief and the patient's risk of a seriously adverse CV event (e.g., myocardial infarction). For example, a patient who has a high risk of infarct prior to COX-2 inhibitor therapy is co-administered VIOXX®/LIPITOR® in a ratio of 0.625:1. A patient who has a low risk of infarct prior to COX-2 inhibitor therapy is co-administered VIOXX®/LIPITOR® in a ratio of 1.25:1.

TABLE 5

| | Moderate dose of VIOXX | High dose of VIOXX |
|---|---|---|
| Lower risk | Vioxx 12.5 mg/Lipitor 10 mg | Vioxx 25 mg/Lipitor 20 mg |
| of infarct | | |
| Higher risk | Vioxx 12.5 mg/Lipitor 20 mg | Vioxx 25 mg/Lipitor 40 mg |
| of infarct | | |

### EXAMPLE 3

### Daily Dosage Regimen for Co-Administration of the COX-2 Inhibitor Valdecoxib (BEXTRA®) with the Statin Atorvastatin (LIPITOR®)

Table 6 provides dosage regimens for the co-administration of valdecoxib with atorvastatin. As illustrated in the Table 4, the ratio of BEXTRA® to LIPITOR® is adjusted based on the patient's need for effective pain relief and the patient's risk of a seriously adverse CV event (e.g., myocardial infarction). For example, a patient who has a high risk of infarct prior to COX-2 inhibitor therapy is co-administered BEXTRA®/LIPITOR® in a ratio of 0.5:1. A patient who has a low risk of infarct prior to COX-2 inhibitor therapy is co-administered BEXTRA®/LIPITOR® in a ratio of 1:1.

TABLE 6

| | Moderate dose of Bextra | High dose of Bextra |
|---|---|---|
| Low risk | Bextra 10 mg/Lipitor 10 mg | Bextra 20 mg/Lipitor 20 mg |
| of infarct | | |
| High risk | Bextra 10 mg/Lipitor 20 mg | Bextra 20 mg/Lipitor 40 mg |
| of infarct | | |

The exemplary pattern of dosages for these three COX-2 inhibitors can be translated, with appropriate modification for equivalent dosages of individual drugs, to other COX-2 inhibitors nonselective NSAIDs and other pharmaceutical agents which induce or increase the risk of an adverse CV event. The exemplary pattern of dosages for LIPITOR® can be translated, with appropriate modification for equivalent dosages of individual drugs, to other statins, to ACE inhibitors and ARBs, and to other pharmaceutical agents which prevent CV events.

The present invention is not limited in scope by the specific embodiments described herein. Indeed, various modifications of the invention in addition to those described herein will become apparent to those skilled in the art from the foregoing description. Such modifications are intended to fall within the scope of the appended claims. It is further to be understood that all values given in the foregoing examples are approximate, and are provided for purposes of illustration.

Patents, patent applications, publications, product descriptions, and protocols which are cited throughout this application are incorporated herein by reference in their entireties for all purposes.

### REFERENCE LIST FOR TABLES 1-3

1. Lefkowitz R J, Hoffman B B, Taylor P. Neurotransmission: the autonomic and somatic motor nervous systems. In: Hardman J G, Limbird L E, Molinoff P B, Ruddon R W, Gilman A G, editors. The Pharmacological Basis of Therapeutics. New York: McGraw-Hill, 1996: 105-139.
2. Hijmering M L, Stroes E S G, Olijhoek J et al. Sympathetic activation markedly reduces endothelium-dependent, flow-mediated vasodilation. J Am Coll Cardiol. 2002; 39:683-688.
3. Cannon W B. Bodily Changes in Pain, Hunger, Fear and Rage. An Account of Recent Researches into the Function of Emotional Excitement. 2nd ed. Boston: Charles T. Branford Co, 1953.
4. Black P H. Stress and the inflammatory response: a review of neurogenic inflammation. Brain Behav Immun. 2002; 16:622-653.
5. Remme W J. The sympathetic nervous system and ischaemic heart disease. Eur Heart J. 1998; 19(suppl F):F62-F71.
6. Julius S, Majahalme S. The changing face of sympathetic overactivity in hypertension. Ann Med. 2000; 32:365-370.
7. Esler M, Rumantir M, Wiesner G et al. Sympathetic nervous system and insulin resistance: from obesity to diabetes. Am J Hypertens. 2001; 14:304S-309S.
8. Verma S, Anderson T J. Fundamentals of endothelial function for the clinical cardiologist. Circulation. 2002; 105:546-549.
9. Iaccarino G, Ciccarelli M, Sorriento D et al. AKT participates in endothelial dysfunction in hypertension. Circulation. 2004; 109:2587-2593.
10. Expert Panel on Detection EaToHBC. Detection, evaluation, and treatment of high blood cholesterol in adults (Adult Treatment Panel III): third report of the National Cholesterol Education Program (NCEP) Expert Panel. Circulation. 2002; 106:3145-3421.
11. Chobanian A V, Bakris G L, Black H R et al. The Seventh Report of the Joint National Committee on Prevention, Detection, Evaluation, and Treatment of High Blood Pressure: the JNC 7 report. JAMA. 2003; 289:2560-2572.
12. Welzig C M, Shin D G, Park H J et al. Lipid lowering by pravastatin increases parasympathetic modulation of heart rate: Gα,₂, a possible marker for parasympathetic responsiveness. Circulation. 2003; 108:2743-2746.
13. De Curtis A, D'Adamo M C, Amore C et al. Experimental arterial thrombosis in genetically or diet induced hyperlipidemia in rats—role of vitamin K-dependent clotting factors and prevention by low-intensity oral anticoagulation. Thromb Haemost. 2001; 86:1440-1448.
14. Hutter R, Valdiviezo C, Sauter B V et al. Caspase-3 and tissue factor expression in lipid-rich plaque macrophages: evidence for apoptosis as link between inflammation and atherothrombosis. Circulation. 2004; 109:2001-2008.
15. Reaven G M, Lithell H, Landsberg L. Hypertension and associated metabolic abnormalities—the role of insulin resistance and the sympathoadrenal system. N Engl J. Med. 1996; 334:374-381.
16. Ridker P M, Glynn R J, Hennekens C H. C-reactive protein adds to the predictive value of total and HDL cholesterol in determining risk of first myocardial infarction. Circulation. 1998; 97:2007-2011.
17. Sesso H D, Buring J E, Rifai N et al. C-reactive protein and the risk of developing hypertension. JAMA. 2003; 290:2945-2951.
18. Pickup J C. Inflammation and activated innate immunity in the pathogenesis of type 2 diabetes. Diabetes Care. 2004; 27:813-823.
19. Esmon C T. Crosstalk between inflammation and thrombosis. Maturitas. 2004; 47:305-314.
20. Ross R. Atherosclerosis—an inflammatory disease. N Engl J Med. 1999; 340:115-126.
21. Grundy S M. Inflammation, hypertension, and the metabolic syndrome. JAMA. 2003; 290:3000-3002.
22. Julius S, Gudbrandsson T. Early association of sympathetic overactivity, hypertension, insulin resistance, and coronary risk. J Cardiovasc Pharmacol. 1992; 20(suppl 8):S40-S48.
23. Boden G, Hoeldtke R D. Nerves, fat, and insulin resistance. N Engl J. Med. 2003; 349:1966-1967.
24. Sobel B E. Insulin resistance and thrombosis: a cardiologist's view. Am J Cardiol. 1999; 84:37J-41J.
25. Morrow L A, Halter J B. Diabetes in the older adult. Geriatrics. 1988; 43(suppl):57-65.
26. Esler M, Hastings J, Lambert G et al. The influence of aging on the human sympathetic nervous system and brain norepinephrine turnover. Am J Physiol. 2002; 282:R909-R916.
27. May O, Arildsen H, Møller M. Parasympathetic function during deep breathing in the general population: relation to coronary risk factors and normal range. J Intern Med. 1999; 245:287-294.
28. Wilkerson W R, Sane D C. Aging and thrombosis. Semin Thromb Hemost. 2002; 28:555-567.
29. Gostynski M, Gutzwiller F, Kuulasmaa K et al. Analysis of the relationship between total cholesterol, age, body mass index among males and females in the WHO MONICA Project. Int J Obes Relat Metab Dis. 2004; 28:1082-1090.
30. Franceschi C, Bonafe M, Valensin S et al. Inflamm-aging: an evolutionary perspective on immunosenescence. Ann N Y Acad Sci. 2000; 908:244-254.
31. Røder ME, Schwartz R S, Prigeon R L et al. Reduced pancreatic B cell compensation to the insulin resistance of aging: impact on proinsulin and insulin levels. J Clin Endocrinol Metab. 2000; 85:2275-2280.
32. Paradis G, Lambert M, O'Loughlin J et al. Blood pressure and adiposity in children and adolescents. Circulation. 2004; 110:1832-1838.
33. Eckel R H, Barouch W W, Ershow A G. Report of the National Heart, Lung, and Blood Institute-National Institute of Diabetes and Digestive and Kidney Diseases Working Group on the pathophysiology of obesity-associated CV disease. Circulation. 2002; 105:2923.
34. Alvarez G E, Beske S D, Ballard T P et al. Sympathetic neural activation in visceral obesity. Circulation. 2002; 106:2533.
35. Pathak A, Smih F, Galinier M et al. Insulin downregulates M(2)-muscarinic receptors in adult rat atrial cardiomyocytes: a link between obesity and cardiovascular complications. Int J Obes Relat Metab Discord. 2005; 29:176-182.
36. Berg A H, Scherer P E. Adipose tissue, inflammation, and CV disease. [Review] [133 refs]. Circ Res. 2005; 96:939-949.
37. Barengo N C, Hu G, Kastarinen M et al. Low physical activity as a predictor for antihypertensive drug treatment in 25-64-year-old populations in eastern and south-western Finland. [see comment]. J Hypertens. 2005; 23:293-299.
38. Sullivan P W, Morrato E H, Ghushchyan V et al. Obesity, inactivity, and the prevalence of diabetes and diabetes-related cardiovascular comorbidities in the U.S., 2000-2002. Diabetes Care. 2005; 28:1599-1603.
39. Hellstrom H R. Evidence in support of the spasm of resistance vessel concept of ischemic heart disease: an update in 1993. Med Hypotheses. 1993; 41:11-22.
40. Goldsmith R L, Bigger J T Jr, Steinman R C et al. Comparison of 24-hour parasympathetic activity in endurance-trained and untrained young men. J Am Coll Cardiol. 1992; 20:552-558.
41. Lee K W, Lip G Y H. Effects of lifestyle on hemostasis, fibinolysis, and platelet reactivity: a systematic review. Arch Intern Med. 2003; 163:2368-2392.
42. Das U N. Anti-inflammatory nature of exercise. Nutrition. 2004; 20:323-326.
43. LaMonte M J, Blair S N, Church T S. Physical activity and diabetes prevention. [Review][84 refs]. J Appl Physiol. 2005; 99:1205-1213.
44. Rozanski A, Blumenthal J A, Kaplan J. Impact of psychological factors on the pathogenesis of CV disease and implications for therapy. Circulation. 1999; 99:2192-2217.
45. Yan L L, Liu K, Matthews K A et al. Psychosocial factors and risk of hypertension: the Coronary Artery Risk Development in Young Adults (CARDIA) study. JAMA. 2003; 290:2138-2148.
46. Rosmond R. Stress induced disturbances of the HPA axis: a pathway to Type 2 diabetes? Med Sci Monit. 2003; 9:RA35-RA39.
47. Black P H, Garbutt L D. Stress, inflammation and CV disease. J Psychosom Res. 2002; 52: 1-23.
48. Mezzacappa E S, Kelsey R M, Katkin E S et al. Vagal rebound and recovery from psychological stress. Psychosom Med. 2001; 63:650-657.
49. Black P H. The inflammatory response is an integral part of the stress response: implications for atherosclerosis, insulin resistance, type II diabetes and metabolic syndrome X. Brain Behav Immun. 2003; 17:350-364.
50. Haire-Joshu D, Glascow R E, Tibbs T L. Smoking and diabetes. Diabetes Care. 1999; 22:1887-1898.
51. Hjemdahl P. Smoking, nicotine and thrombotic risk—a role for platelet dependent thrombin generation? Eur Heart J. 2001; 22:16-18.
52. Tsiara S, Elisaf M, Mikhailidis D P. Influence of smoking on predictors of vascular disease. Angiology. 2003; 54:507-530.
53. Eliasson B. Cigarette smoking and diabetes. Prog Cardiovasc Dis. 2003; 45:405-413.
54. Manfredini R, Gallerani M, Portaluppi F et al. Relationships of the circadian rhythms of thrombotic, ischemic, hemorrhagic, and arrhythmic events to blood pressure rhythms. Ann N Y Acad Sci. 1996; 783:141-158.
55. la Fleur S E, Kalsbeek A, Wortel J et al. A daily rhythm in glucose tolerance: a role for the suprachiasmatic nucleus. Diabetes. 2001; 50:1237-1243.
56. Trotti R, Rondanelli M, Cuzzoni G et al. Circadian temporal organization of lipidic fractions in elderly people: entrainment to the dietary schedule. Aging Clin Exp Res. 2002; 14:94-99.
57. Panzer S E, Dodge A M, Kelly E A B et al. Circadian variation of sputum inflammatory cells in mild asthma. J Allergy Clin Immunol. 2003; 111:308-312.
58. Spencer F A, Goldberg R J, Becker R C et al. Seasonal distribution of acute myocardial infarction in the second National Registry of Myocardial Infarction. J Am Coll Cardiol. 1998; 31:1226-1233.
59. Sun Z, Wang X, Wood C E et al. Genetic ATIA receptor deficiency attenuates cold-induced hypertension. Am Journal Physiol. 2005; 288:R433-R439.
60. Tseng C L, Brimacombe M, Xie M et al. Seasonal patterns in monthly hemoglobin AIc values. Am J Epidemiology. 2005; 161:565-574.
61. Kawahara J, Sano H, Fukuzaki H et al. Acute effects of exposure to cold on blood pressure, platelet function and sympathetic nervous activity in humans. Am J Hypertens. 1989; 2:724-726.
62. Gyllerup S, Lanke J, Lindholm L H et al. Cold climate is an important factor in explaining regional differences in coronary mortality even if serum cholesterol and other established risk factors are taken into account. Scot Med J. 1993; 38:169-172.
63. Mercer J B, Østerud B, Tveita T. The effect of short-term cold exposure on risk factors for CV disease. Thromb Res. 1999; 95:93-104.
64. Torsoni M A, Carvalheira J B, Pereira-Da-Silva M et al. Molecular and functional resistance to insulin in hypothalamus of rats exposed to cold. Am J Physiol. 2003; 285:E216-E223.
65. Hayden M, Pignone M, Phillips C et al. Aspirin for the primary prevention of cardiovascular events: a summary of the evidence for the U.S. Preventive Services Task Force. Ann Intern Med. 2002; 136:161-172.
66. Pfeffer M A, McMurray J J V, Velazquez E J et al. Valsartan, captopril, or both in myocardial infarction complicated by heart failure, left ventricular dysfunction, or both. N Engl J Med. 2003; 349: 1893-1906.
67. Chiquette E, Ramirez G, DeFronzo R. A meta-analysis comparing the effect of thiazolidinediones on CV risk factors. Arch Intern Med. 2004; 164:2097-2104.
68. Michels K B, Manson J E. Postmenopausal hormone therapy: a reversal of fortune. Circulation. 2003; 107:1830-1833.
69. Bybee K A, Kopecky S L, Williams B A et al. Reduced creatine kinase release with statin use at the time of myocardial infarction. Int J Cardiol. 2004; 96:461-466.
70. Brouwer M A, van den Bergh P J P C, Aengevaeren W R M et al. Aspirin plus coumarin versus aspirin alone in the prevention of reocclusion after fibrinolysis for acute myocardial infarction: results of the Antithrombotics in the Prevention of Reocclusion In Coronary Thrombolysis (APRICOT)-2 Trial. Circulation. 2002; 106:659-665.
71. Takagi T, Akasaka T, Yamamuro A et al. Troglitazone reduces neointimal tissue proliferation after coronary stent implantation in patients with non-insulin dependent diabetes mellitus: a serial intravascular ultrasound study. J Am Coll Cardiol. 2000; 36:1529-1535.
72. Golomb B A, Ritchie J B, Criqui M H et al. Statins lower blood pressure: results from the UCSD statin study. Circulation. 2004; 110 (suppl III):III-402.
73. Wald N J, Law M R. A strategy to reduce CV disease by more than 80%. BMJ. 2003; 326:1419-1422.
74. Dobrian A D, Schriver S D, Khraibi A A et al. Pioglitazone prevents hypertension and reduces oxidative stress in diet-induced obesity. Hypertension. 2004; 43:48-56.
75. Wyss J M, Carlson S H. Effects of hormone replacement therapy on the sympathetic nervous system and blood pressure. Curr Hypertens Rep. 2003; 5:241-246.
76. Magen E, Viskoper R, Mishal J et al. Resistant arteral hypertension and hyperlipidemia: atorvastatin, not vitamin C, for blood pressure control. Isr Med Assoc J. 2004; 6:742-746.
77. Hermida R C, Ayala D E, Calvo C et al. Administration time-dependent effects of aspirin on blood pressure in untreated hypertensive patients. Hypertension. 2003; 41:1259-1267.
78. Freeman D J, Norrie J, Sattar N et al. Pravastatin and the development of diabetes mellitus: evidence for a protective treatment effect in the West of Scotland Coronary Prevention Study. Circulation. 2001; 103:357-362.
79. Scheen A J. Prevention of type 2 diabetes mellitus through inhibition of the renin-angiotensin system. Drugs. 2004; 64:2537-2565.
80. Buchanan T A, Xiang A H, Peters R K et al. Preservation of pancreatic β-cell function and prevention of type 2 diabetes by pharmacological treatment of insulin resistance in high-risk hispanic women. Diabetes. 2002; 51:2796-2803.
81. Kanaya A M, Herrington D, Vittinghoff E et al. Glycemic effects of postmenopausal hormone therapy: the Heart and Estrogen/progestin Replacement Study: a randomized, double-blind, placebo-controlled trial. Ann Int Med. 2003; 138:1-9.
82. Paniagua J A, Löpez-Miranda J, Escribano A et al. Cerivastatin improves insulin sensitivity and insulin secretion in early-state obese type 2 diabetes. Diabetes. 2002; 51:2596-2603.
83. The Heart Outcomes Prevention Evaluation Study Investigators. Effects of an angiotensin-converting-enzyme inhibitor, ramipril, on cardiovascular events in high-risk patients. N Engl J Med. 2000; 342: 145-153.
84. Yki-Järvinen H. Thiazolidinediones. N Eng J Med. 2004; 351:1106-1118.
85. Abelson K S P, Kommalage M, Hoglund U. Spinal cholinergic involvement after treatment with aspirin and paracetamol in rats. Neurosci Lett. 2004; 368:116-120.
86. Kontopoulos A G, Athyros V G, Papageorgiou A A et al. Effect of quinapril or metoprolol on circadian sympathetic and parasympathetic modulation after acute myocardial infarction. Am J Cardiol. 1999; 84:1164-1169.
87. Manhem K, Brandin L, Ghanoum B et al. Acute effects of transdermal estrogen on hemodynamic and vascular reactivity in elderly postmenopausal healthy women. J Hypertens. 2003; 21:387-394.
88. Kishi T, Hirooka Y, Mukai Y et al. Atorvastatin causes depressor and sympatho-inhibitory effects with upregulation of nitric oxide synthases in stroke-prone spontaneously hypertensive rats. J Hypertens. 2003; 21:379-386.
89. Nagai Y, Sugiyama Y, Nakatsumi Y et al. Rapid amelioration of muscle sympathetic nerve activity by pioglitazone in an obese Type 2 diabetic patient. Diabet Med. 2003; 20:868-869.
90. Vongpatanasin W, Tuncel M, Mansour Y et al. Transdermal estrogen replacement therapy decreases sympathetic activity in postmenopausal women. Circulation. 2001; 103:2903-2908.
91. Quyyumi A A. Effects of aspirin on endothelial dysfunction in atherosclerosis. Am J Cardiol. 1998; 82:31S-33S.
92. Sidhu J S, Cowan D, Kaski J-C. The effects of rosiglitazone, a peroxisome proliferator-activated receptor-gamma agonist, on markers of endothelial cell activation, C-reactive protein, and fibrinogen levels in non-diabetic coronary artery disease patients. J Am Coll Cardiol. 2003; 42:1757-1763.
93. Rosenson R S, Tangney C C. Antiatherothrombotic properties of statins: implications for cardiovascular event reduction. JAMA. 1998; 279:1643-1650.
94. Chiang N, Bermudez E A, Ridker P M et al. Aspirin triggers antiinflammatory 15-epi-lipoxin A4 and inhibits thromboxane in a randomized human trial. PNAS. 2004; 101:15178-15183.
95. Gryglewski R J, Swies J, Uracz W et al. Mechanisms of angiotensin-converting enzyme inhibitor induced thrombolysis in Wistar rats. Thromb Res. 2003; 110:323-329.
96. Rosendaal F R, Helmerhorst F M, Vandenbroucke J P. Female hormones and thrombosis. Arterioscler Thromb Vasc Biol. 2002; 22:201-210.
97. Hundal R S, Petersen K F, Mayerson A B et al. Mechanism by which high-dose aspirin improves glucose metabolism in type 2 diabetes. J Clin Invest. 2002; 109:1321-1326.
98. Kon V, Jabs K. Angiotensin in atherosclerosis. Curr Opin Nephrol Hypertens. 2004; 13:291-297.
99. Khan M, Xu Y, Edwards G et al. Effects of pioglitazone on the components of diabetic dyslipidaemia: results of double-blind, multicentre, randomised studies. Int J Clin Pract. 2004; 58:907-912.
100. Faludi A A, Aldrighi J M, Bertolami M C et al. Progesterone abolishes estrogen and/or atorvastatin endothelium dependent vasodilatory effects. Atherosclerosis. 2004; 177:89-96.
101. Lefer D J. Statins as potent antiinflammatory drugs. Circulation. 2002; 106:2041-2042.
102. Hsuch W A, Lyon C J, Quiñones M J. Insulin resistance and the endothelium. Am J Med. 2004; 117:109-117.
103. Thomas T N, Rhodin J A, Clark L et al. A comparison of the anti-inflammatory activities of conjugated estrogens and 17-β estradiol. Inflamm Res. 2003; 52:452-460.
104. Greenfield J R, Samaras K, Jenkins A B et al. Moderate alcohol consumption, estrogen replacement therapy, and physical activity are associated with increased insulin sensitivity: is abdominal adiposity the mediator? Diabetes Care. 2003; 26:2734-2740.
105. Starr J M, McGurn B, Whiteman M et al. Life long changes in cognitive ability are associated with prescribed medications in old age. Int J Geriatr Psychiatry. 2004; 327-332.
106. Shepherd C E, Piguet O, Broe G A et al. Histocompatibility antigens, aspirin use and cognitive performance in non-demented elderly subjects. J Neuroimmunol. 2004; 148:178-182.
107. Tedesco M A, Ratti G, Di Salvo G et al. Does the angiotensin II receptor antagonist losartan improve cognitive function? Drugs Aging. 2002; 19:723-732.
108. Fillit H M, Butler R N, O'Connell A W et al. Achieving and maintaining cognitive vitality with aging. Mayo Clin Proc. 2002; 77:681-696.
109. Lupattelli G, Scarponi A M, Vaudo G et al. Simvastatin increases bone mineral density in hypercholesterolemic postmenopausal women. Metabolism. 2004; 53:744-748.
110. Carbone L D, Tylavsky F A, Cauley J A et al. Association between bone mineral density and the use of nonsteroidal anti-inflammatory drugs and aspirin: impact of cyclooxygenase selectivity. J Bone Miner Res. 2003; 18:1795-1802.
111. Hamerman D. Osteoporosis and atherosclerosis: biological linkages and the emergence of dual-purpose therapies. Q J Med. 2005; 98:467-484.
112. Clemons M, Goss P. Estrogen and the risk of breast cancer. N Engl J Med. 2001; 344:276-285.
113. Rea T D, Breitner J C, Psaty B M et al. Statin use and the risk of incident dementia. Arch Neurol. 2005; 62: 1047-1051.
114. Broe G A, Grayson D A, Creasey H M et al. Anti-inflammatory drugs protect against Alzheimer disease at low doses. Arch Neurol. 2000; 57:1586-1591.
115. Combs C K, Johnson D E, Karlo J C et al. Inflammatory mechanisms in Alzheimer's disease: inhibition of β-amyloid-stimulated proinflammatory responses and neurotoxicity by PPARγ agonists. J Neurosci. 2000; 20:558-567.
116. Zandi P P, Carlson M C, Plassman B L et al. Hormone replacement therapy and incidence of Alzheimer Disease in older women: the Cache County Study. JAMA. 2002; 288:2123-2129.
117. Young-Xu Y, Jabbour S, Goldberg R et al. Usefulness of statin drugs in protecting against atrial fibrillation in patients with coronary artery disease. Am J Cardiol. 2003; 92: 1379-1383.
118. Healey J S, Baranchuk A, Crystal E et al. Prevention of atrial fibrillation with angiotensin-converting enzyme inhibitors and angiotensis receptor blockers: a meta-analysis. J Am Coll Cardiol. 2005; 45:1832-1839.
119. Poynter J N, Gruber S B, Higgins P D R et al. Statins and the risk of colorectal cancer. N Engl J Med. 2005; 352:2184-2192.
120. Gonzalez-Perez A, Garcia Rodriguez L A, Lopez-Ridaura R. Effects of non-steroidal anti-inflammatory drugs on cancer sites other than the colon and rectum: a meta-analysis. BMC Cancer. 2003; 3:28.
121. Lindberg H, Nielsen D, Jensen B V et al. Angiotensin converting enzyme inhibitors for cancer treatment? Acta Oncol. 2004; 43:142-152.
122. Nahle Z. PPAR trilogy from metabolism to cancer. Curr Opin Clin Nutr Metab Care. 2004; 7:397-402.
123. Thompson P D, Buchner D, Piña I L et al. Exercise and physical activity in the prevention and treatment of atherosclerotic CV disease: a statement from the Council on Clinical Cardiology (Subcommittee on Exercise, Rehabilitation, and Prevention) and the Council on Nutrition, Physical Activity, and Metabolism (Subcommittee on Physical Activity). Circulation. 2003; 107:3109-3116.
124. de Lorgeril M, Salen P. Is alcohol anti-inflammatory in the context of coronary heart disease? Heart. 2004; 90:355-357.
125. He K, Song Y, Daviglus M L et al. Accumulated evidence on fish consumption and coronary heart disease mortality: a meta-analysis of cohort studies. Circulation. 2004; 109:2705-2711.
126. Ros E, Núñez I, Perez-Heras A et al. A walnut diet improves endothelial function in hypercholesterolemic subjects: a randomized crossover trial. Circulation. 2004; 109:1609-1614.
127. de Lorgeril M, Salen P, Martin J-L et al. Mediterranean diet, traditional risk factors, and the rate of cardiovascular complications after myocardial infarction: final report of the Lyon Diet Heart Study. Circulation. 1999; 99:779-785.
128. Sies H, Schewe T, Heiss C et al. Cocoa polyphenols and inflammatory mediators. Am J Clin Nutr. 2005; 81 (suppl):304S-312S.
129. GISSI-Prevenzione Investigators. Dietary supplementation with n-3 polyunsaturated fatty acids and vitamin E after myocardial infarction: results of the GISSI-Prevenzione trial. Lancet. 1999; 354:447-455.
130. Franco V, Oparil S, Carretero O A. Hypertensive therapy: part II. Circulation. 2004; 109:3081-3088.
131. Matthews K A, Katholi C R, McCreath H et al. Blood pressure reactivity to psychological stress predicts hypertension in the CARDIA study. Circulation. 2004; 110:74-78.
132. Psaltopoulou T, Naska A, Orfanos P et al. Olive oil, the Mediterranean diet, and arterial blood pressure: the Greek European Prospective Investigation into Cancer and Nutrition (EPIC) study. Am J Clin Nutr. 2004; 80:1012-1018.
133. Al-Awwadi N A, Bornet A, Azay J et al. Red wine polyphenols alone or in association with ethanol prevent hypertension, cardiac hypertrophy, and production of reactive oxygen species in the insulin-resistant fructose-fed rat. J Agric Food Chem. 2004; 52:5593-5597.
134. Krantz D S, McCeney M K. Effects of psychological and social factors on organic disease: a critical assessment of research on coronary heart disease. Annu Rev Psychol. 2002; 53:341-369.
135. Geleijnse J M, Giltay E J, Grobbee D E et al. Blood pressure response to fish oil supplementation: metaregression analysis of randomized trials. J Hypertens. 1920; 1493-1499.
136. Sjöström L, Lindroos A-K, Peltonen M et al. Lifestyle, diabetes, and cardiovascular rsk factors 10 years after bariatric surgery. N Eng J Med. 2004; 351:2683-2693.
137. Wales J K. Does psychological stress cause diabetes? Diabet Med. 1995; 12:109-112.
138. Howard A A, Arnsten J H, Gourevitch M N. Effect of alcohol consumption on diabetes mellitus: a systematic review. Ann Intern Med. 2004; 140:211-219.
139. Jiang R, Manson J E, Stampfer M J et al. Nut and peanut butter consumption and risk of type 2 diabetes in women. JAMA. 2002; 288:2554-2560.
140. Biesalski H K. Diabetes preventive components in the Mediterranean diet. Eur J Nutr. 2004; 43(suppl 1):126-130.
141. Inui A, Kitaoka H, Majima M et al. Effect of the Kobe earthquake on stress and glycemic control in patients with diabetes mellitus. Arch Intern Med. 1998; 158:274-278.
142. Goldsmith R L, Bloomfield D M, Rosenwinkel E T. Exercise and autonomic function. Coron Artery Dis. 2000; 11:129-135.
143. Laaksonen D E, Laitinen T, Schönberg J et al. Weight loss and weight maintenance, ambulatory blood pressure and cardiac autonomic tone in obese persons with the metabolic syndrome. J Hypertens. 2003; 21:371-378.
144. Sakakibara M, Takeuchi S, Hayano J. Effect of relaxation training on cardiac parasympathetic tone. Psychophysiology. 1994; 31:223-228.
145. Murata K, Araki S, Yokoyama K et al. Autonomic neurotoxicity of alcohol assessed by heart rate variability. J Auton Nerv Syst. 1994; 48:105-111.
146. Das U N. Beneficial effect(s) of n-3 fatty acids in CV diseases: but, why and how? Prostaglandins Leukot Essent Fatty Acids. 2000; 63:351-362.
147. Trombetta I C, Batalha L T, Rondon M U P B et al. Weight loss improves neurovascular and muscle metaboreflex control in obesity. Am J Physiol. 2003; 285:H974-H982.
148. Lucini D, Covacci G, Milani R et al. A controlled study of the effects of mental relaxation on autonomic excitatory responses in healthy subjects. Psychosom Med. 1997; 59:541-552.
149. van de Borne P, Mark A L, Montano N et al. Effects of alcohol on sympathetic activity, hemodynamics, and chemoreflex sensitivity. Hypertension. 1997; 29:1278-1283.
150. Jennings G L R, Chin-Dusting J P F, Kingwell B A et al. Modulation of vascular function by diet and exercise. Clin Exp Hypertens. 1997; 19:727-737.
151. Higashi Y, Sasaki S, Kurisu S et al. Regular aerobic exercise augments endothelium-dependent vascular relaxation in normotensive as well as hypertensive subjects: role of endothelium-derived nitric oxide. Circulation. 1999; 100:1194-1202.
152. Ziccardi P, Nappo F, Giugliano G et al. Reduction of inflammatory cytokine concentrations and improvement of endothelial functions in obese women after weight loss over one year. Circulation. 2002; 105:804-809.
153. Spieker L E, Hürlimann D, Ruschitzka F et al. Mental stress induces prolonged endothelial dysfunction via endothelin-A receptors. Circulation. 2002; 105:2817-2820.
154. Teragawa H, Fukuda Y, Matsuda K et al. Effect of alcohol consumption on endothelial function in men with coronary artery disease. Atherosclerosis. 2002; 165:145-152.
155. Esposito K, MARBella R, Ciotola M et al. Effect of a Mediterranean-style diet on endothelial dysfunction and markers of vascular inflammation in the metabolic syndrome. JAMA. 2004; 292:1440-1446.
156. Lacoste L, Hung J, Lam J Y T. Acute and delayed antithrombotic effects of alcohol in humans. Am J Cardiol. 2001; 87:82-85.
157. Mruk J S, Webster M W I, Heras M et al. Flavone-8-acetic acid (flavonoid) profoundly reduces platelet-dependent thrombosis and vasoconstriction after deep arterial injury In vivo. Circulation. 2000; 101:324-328.
158. Klein S, Burke L E, Bray G A et al. Clinical implications of obesity with specific focus on cadiovascular disease. A statement for professional from the American Heart Association Council on Nutrition, Physical Activity, and Metabolism. Circulation. 2004; 110:2952-2967.
159. Wannamethee S G, Lowe G D O, Shaper G et al. The effects of different alcoholic drinks on lipids, insulin and haemostatic and inflammatory markers in older men. Thromb Haemost. 2003; 90:1080-1087.
160. Tapsell L C, Gillen L J, Patch C S et al. Including walnuts in a low-fat/modified-fat diet improves HDL cholesterol-to-total cholesterol ratios in patients with type 2 diabetes. Diabetes Care. 2004; 27:2777-2783.
161. Chiba H, Uehara M, Wu J et al. Hesperidin, a citrus flavonoid, inhibits bone loss and decreases serum and hepatic lipids in ovariectomized mice. J Nutr. 2003; 133:1892-1897.
162. Kasapis C, Thompson P D. The effects of physical activity on serum C-reactive protein and inflammatory markers: a systematic review. J Am Coll Cardiol. 2005; 45:1563-1569.
163. Lutgendorf S, Logan H, Kirchner H L et al. Effects of relaxation and stress on the capsaicin-induced local inflammatory response. Psychosom Med. 2000; 62:524-534.
164. Albert M A, Glynn R J, Ridker P M. Alcohol consumption and plasma concentration of C-reactive protein. Circulation. 2003; 107:443-447.
165. Raikkonen K, Keltikangas-Jarvinen L, Adlercreutz H et al. Psychosocial stress and the insulin resistance syndrome. Metabolism. 1996; 45:1533-1538.
166. Sirtori C R, Galli C. N-3 fatty acids and diabetes. Biomed Pharmacother. 2002; 56:397-406.
167. Garcia-Lorda P, Megias R, I, Salas-Salvado J. Nut consumption, body weight and insulin resistance. Eur J Clinl Nutr. 2003; 57(suppl 1):S8-S11.
168. Weuve J, Kang J H, Manson J E et al. Physical activity, including walking, and cognitive function in older women. JAMA. 2004; 292:1454-1461.
169. Bagger Y Z, Tanko L B, Alexandersen P et al. The implications of body fat mass and fat distribution for cognitive function in elderly women. Obes Res. 2004; 12:1519-1526.
170. Stampfer M J, Kang J H, Chen J et al. Effects of moderate alcohol consumption on cognitive function in women. N Engl J Med. 2005; 352:245-253.
171. Kalmijn S. Fatty acid intake and the risk of dementia and cognitive decline: a review of clinical and epidemiological studies. J Nutr Health Aging. 2000; 4:202-207.
172. Joseph J A, Shukitt-Hale B, Casadesus G. Reversing the deleterious effects of aging on neuronal communication and behavior: beneficial properties of fruit polyphenolic compounds. Am J Clin Nutr. 2005; 81(suppl):313S-316S.
173. MacKelvie K J, Petit M A, Khan K M et al. Bone mass and structure are enhanced following a 2-year randomized controlled trial of exercise in prepubertal boys. Bone. 2004; 34:755-764.
174. Guney E, Kisakol G, Ozgen G et al. Effect of weight loss on bone metabolism: comparison of vertical banded gastroplasty and medical intervention. Obes Surg. 2003; 13:383-388.
175. Bakhireva L N, Barrett-Connor E, Kritz-Silverstein D et al. Modifiable predictors of bone loss in older men: a prospective study. Am J Prev Med. 2004; 26:436-442.
176. Judex S, Wohl G R, Wolff R B et al. Dietary fish oil supplementation adversely affects cortical bone morphology and biomechanics in growing rabbits. Calcif Tissue Int. 2000; 66:443-448.
177. Abbott R D, White L R, Ross G W et al. Walking and dementia in physically capable elderly men. JAMA. 2004; 292:1447-1453.
178. Wilson R S, Evans D A, Bienias J L et al. Proneness to psychological distress is associated with risk of Alzheimer's disease. Neurology. 2003; 61:1479-1485.
179. Luchsinger J A, Tang M X, Siddiqui M et al. Alcohol intake and risk of dementia. J Am Geriatr Soc. 2004; 52:540-546.
180. Morris M C, Evans D A, Bienias J L et al. Consumption of fish and n-3 fatty acids and risk of incident Alzheimer disease. Arch Neurol. 2003; 60:940-946.
181. Panza F, Solfrizzi V, Colacicco A M et al. Mediterranean diet and cognitive decline. Public Health Nutr. 2004; 7:959-963.
182. Weinreb O, Mandel S, Amit T et al. Neurological mechanisms of green tea polyphenols in Alzheimer's and Parkinson's diseases. J Nutr Biochem. 2004; 15:506-516.
183. Houghton J L, Devlin C W, Besson W T I et al. Possible triggering of paroxysmal atrial fibrillation in normal hearts by psychological stressors: a report of two cases. Am J Med Sci. 1990; 300:234-236.
184. Frost L, Vestergaard P. Alcohol and risk of atrial fibrillation or flutter: a cohort study. Arch Intern Med. 2004; 164:1993-1998.
185. Mozaffarian D, Psaty B M, Rimm E B et al. Fish intake and risk of incident atrial fibrillation. Circulation. 2004; 110:368-373.
186. Alfano C M, Klesges R C, Murray D M et al. Physical activity in relation to all-site and lung cancer incidence and mortality in current and former smokers. Cancer Epidemiol Biomarkers Prev. 2004; 13:2233-2241.
187. Kuriyama S, Tsubono Y, Hozawa A et al. Obesity and risk of cancer in Japan. Int Journal Cancer. 2005; 113:148-157.
188. Reiche E M, Nunes S O, Morimoto H K. Stress, depression, the immune system, and cancer. Lancet Oncol. 2004; 5:617-625.
189. Bianchini F, Vainio H. Wine and resveratrol: mechanisms of cancer prevention? Eur J Cancer Prev. 2003; 12:417-425.
190. Visioli F, Grande S, Bogani P et al. The role of antioxidants in the Mediterranean diets: focus on cancer. Eur J Cancer Prev. 2004; 13:337-343.
191. Neuhouser M L. Dietary flavonoids and cancer risk: evidence from human population studies. Nutr Cancer. 2004; 50:1-7.

What is claimed is:

1. A method for reducing the risk of one or more adverse CV events in a patient to be treated with one or more first pharmaceutical agents associated with inducing or increasing the risk of an adverse CV event, the method comprising administering a pharmaceutical combination comprising:
(a) a therapeutically effective amount of one or more first pharmaceutical agents; and
(b) one or more preventative pharmaceutical agents in an amount effective to reduce the risk of one or more CV events.

2. A method for treating a chronic disorder with one or more first pharmaceutical agents that induce or increase the risk of an adverse CV event, while reducing the patient's risk of an adverse CV event, the method comprising repeatedly co-administering:
(a) a therapeutically effective amount of one or more first pharmaceutical agents; and
(b) one or more preventative pharmaceutical agents in an amount effective to reduce the risk of one or more CV events;
for at least two days.

3. The method of claim 1, wherein:
(a) the one or more first pharmaceutical agents comprises an NSAID, with the proviso that the NSAID is not aspirin; and
(b) the one or more preventative pharmaceutical agents is selected from the group consisting of statins, ARBs, ACE inhibitors, PPAR agents, vasodilators and thiazides.

4. The method of claim 1, wherein the one or more first pharmaceutical agents are selected from the group consisting of SERMs, acetaminophen, muraglitazar, and sympathomimetic agents.

5. The method of claim 4, wherein the sympathomimetic agents are selected from the group consisting of amphetamine aspartate, amphetamine sulfate, dextroamphetamine saccharate, dextroamphetamine sulfate, methylphenidate, dextroamphetamine, ephedrine and atomoxetine.

6. The method of claim 4, wherein the SERM is raloxifene.

7. The method of claim 1, further comprising a recommendation that the patient practice healthy living habits.

8. The method of claim 1, wherein the one or more first pharmaceutical agents and one or more preventative pharmaceutical agents are administered as a unit dosage form.

9. The method of claim 1, wherein the patient is otherwise healthy.

10. A method for reducing the risk of one or more adverse CV events in a patient to be treated with a COX-2 inhibitor, the method comprising administering a pharmaceutical combination comprising:
(a) a therapeutically effective amount of a COX-2 inhibitor; and
(b) one or more preventative pharmaceutical agents in an amount effective to reduce the risk of one or more CV events.

11. The method of claim 10, wherein the COX-2 inhibitor is selected from the group consisting of celecoxib, rofecoxib, valdecoxib, etoricoxib and lumaricoxib.

12. The method of claim 10, wherein the one or more preventative pharmaceutical agents is selected from the group consisting of statins, ARBs, ACE inhibitors, PPAR agents, vasodilators and thiazides.

13. The method of claim 12, wherein the preventative pharmaceutical agent is selected from the group consisting of atorvastatin, lovastatin, pravastatin, simvastatin, rosuvastatin and fluvastatin.

14. A method for reducing the risk of one or more adverse CV events in a patient to be treated with a COX-2 inhibitor, the method comprising administering a pharmaceutical combination comprising:
(a) a therapeutically effective amount of a COX-2 inhibitor selected from the group consisting of celecoxib, rofecoxib, valdecoxib, etoricoxib and lumaricoxib; and
(b) a statin in an amount effective to reduce the risk of one or more CV events.

15. A pharmaceutical composition for reducing the risk of one or more adverse CV events in a patient to be treated with one or more first pharmaceutical agents associated with inducing or increasing the risk of an adverse CV event, the composition comprising a unit dosage form comprising:
(a) a therapeutically effective amount of one or more first pharmaceutical agents; and
(b) one or more preventative pharmaceutical agents in an amount effective to reduce the risk of one or more CV events.

16. The pharmaceutical composition of claim 15, wherein:
(a) the one or more first pharmaceutical agents comprises a COX-2 inhibitor; and
(b) the one or more preventative pharmaceutical agents is selected from the group consisting of statins, ARBs, ACE inhibitors, PPAR agents, vasodilators and thiazides.

17. The pharmaceutical composition of claim 16, wherein:
(a) the COX-2 inhibitor is selected from the group consisting of celecoxib, rofecoxib, valdecoxib, etoricoxib and lumaricoxib; and
(b) the one or more preventative pharmaceutical agents is selected from the group consisting of atorvastatin, lovastatin, pravastatin, simvastatin, rosuvastatin and fluvastatin.

18. The pharmaceutical composition of claim 15, wherein:
(a) the one or more first pharmaceutical agents comprises a SERM; and
(b) the one or more preventative pharmaceutical agents is selected from the group consisting of statins, ARBs, ACE inhibitors, PPAR agents, vasodilators and thiazides.

19. The pharmaceutical composition of claim 18, wherein the SERM is raloxifene.

20. The method of claim 2, wherein repeated co-administration is carried out for at least ten days.
